(19) United States
(12) Patent Application Publication
(10) Pub. No.: US 2026/0209857 A1
(43) Pub. Date: Jul. 23, 2026

(54) METHODS AND COMPOSITIONS FOR ASSESSING COLORECTAL CANCER

(71) Applicant: Singlera Genomics Inc., La Jolla, CA (US)

(72) Inventors: Athurva J. GORE, La Jolla, CA (US); Jeffrey A. GOLE, La Jolla, CA (US); Rui LIU, La Jolla, CA (US)

(73) Assignee: Singlera Genomics Inc., La Jolla, CA (US)

(21) Appl. No.: 19/141,700

(22) PCT Filed: Dec. 20, 2023
(86) PCT No.: PCT/US2023/085219

§ 371 Date: Jun. 20, 2025

Related U.S. Application Data
(60) Provisional application No. 63/434,721, filed on Dec. 22, 2022.

(51) Int. Cl. C12Q 1/6886

(52) U.S. Cl. CPC C12Q 1/6886; C12Q 2600/118; C12Q 2600/154

(57) ABSTRACT

The present disclosure relates to certain compositions, kits, devices, systems and methods, e.g., certain compositions, kits, devices, systems and methods for assessing methylation status, and/or assessing colorectal cancer and/or advanced adenoma in a subject. In particular aspects, provided herein are compositions, kits, devices, systems and methods for assessing colorectal cancer and/or advanced adenoma in a subject based on assessing methylation status of selected target polynucleotide sequences, e.g., target genomic DNA sequences, from the subject.

### RELATED APPLICATION

The present application claims priority to U.S. provisional patent application No. 63/434,721, filed on Dec. 22, 2022, the disclosure of which is incorporated by reference in its entirety for all purposes.

### TECHNICAL FIELD

The present disclosure relates to certain compositions, kits, devices, systems and methods, e.g., compositions, kits, devices, systems and methods for assessing methylation status, and/or assessing colorectal cancer and/or advanced adenoma in a subject. In particular aspects, provided herein are compositions, kits, devices, systems and methods for assessing colorectal cancer or advanced adenoma in a subject based on assessing methylation status of selected target polynucleotide sequences, e.g., target genomic DNA sequences, from the subject.

### BACKGROUND

Various reagents, kits and methods for assessing cancer or neoplasia in a subject are known. However, there is a need for improved compositions, kits, devices, systems and methods for assessing cancer or advanced adenoma in a subject, e.g., tests with better sensitivity, specificity and/or providing more information. The present disclosure addresses this and other related needs.

### BRIEF SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

In one aspect, provided herein is a panel of isolated polynucleotides comprising, consisting of, or consisting essentially of one or at least two isolated polynucleotides, each of said isolated polynucleotides having a polynucleotide sequence of any of Target 1 to Target 83 listed in Table 1, or a complementary or substantially complementary sequence thereof. A kit, device, system or an article of manufacture that comprises any of the above panels is also provided.

In another aspect, a kit, device, system or an article of manufacture that is configured for assessing methylation status of one or at least two of Target 1 to Target 83 listed in Table 1 is provided. In some embodiments, the present kit, device, system or an article of manufacture is configured for assessing colorectal cancer or advanced adenoma in a subject.

In still another aspect, provided herein is an in vitro method for classifying a blood sample, which method comprises: a) in vitro assessing methylation status of one or at least two target polynucleotides in a blood sample from a subject, each of said target polynucleotides having a polynucleotide sequence of any of Target 1 to Target 83 listed in Table 1, or a complementary or substantially complementary sequence thereof; and b) classifying said blood sample into a category based on said methylation status of said one or at least two target polynucleotides in said blood sample obtained in a). In some embodiments, the present methods are used for assessing colorectal cancer or advanced adenoma in a subject. In some embodiments, the present methods are used for diagnosis, prognosis, stratification, risk assessment, or treatment monitoring of colorectal cancer or advanced adenoma in a subject.

### BRIEF DESCRIPTION OF THE DRAWINGS

FIG. 1 illustrates an exemplary support vector machines analysis. Image of FIG. 1 is taken from Wikipedia: https://en.wikipedia.org/wiki/Support_vector_machine), and does not represent any DNA methylation status analysis or cancer assessment.

FIG. 2 illustrates steps for constructing a single-stranded polynucleotide library and performing sequencing analysis using the library, according to one aspect of the present disclosure.

FIG. 3 illustrates a construct comprising a target molecule for sequencing, according to one aspect of the present disclosure.

FIG. 4 illustrates the Bash Code used in Example 1.

FIG. 5 illustrates the Python Code used in Example 2.

FIG. 6 illustrates the Python Code used in Example 4.

FIG. 7 illustrates the Python Code used in Example 5.

### DETAILED DESCRIPTION

Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can, be applied, alone or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

All publications, including patent documents, scientific articles and databases, referred to in this application are incorporated by reference in their entireties for all purposes to the same extent as if each individual publication were individually incorporated by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

The practice of the provided embodiments will employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polypeptide and protein synthesis and modification, polynucleotide synthesis and modification, polymer array synthesis, hybridization and ligation of polynucleotides, detection of hybridization, and nucleotide sequencing. Specific illustrations of suitable techniques can be had by reference to the examples herein. However, other equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, Gabriel, Stephens, Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Ausubel et al. eds., Current Protocols in Molecular Biology (1987); T. Brown ed., Essential Molecular Biology (1991), IRL Press; Goeddel ed., Gene Expression Technology (1991), Academic Press; A. Bothwell et al. eds., Methods for Cloning and Analysis of Eukaryotic Genes (1990), Bartlett Publ.; M. Kriegler, Gene Transfer and Expression (1990), Stockton Press; R. Wu et al. eds., Recombinant DNA Methodology (1989), Academic Press; M. McPherson et al., PCR: A Practical Approach (1991), IRL Press at Oxford University Press; Stryer, Biochemistry (4th Ed.) (1995), W. H. Freeman, New York N.Y.; Gait, Oligonucleotide Synthesis: A Practical Approach (2002), IRL Press, London; Nelson and Cox, Lehninger, Principles of Biochemistry (2000) 3rd Ed., W. H. Freeman Pub., New York, N.Y.; Berg, et al., Biochemistry (2002) 5th Ed., W. H. Freeman Pub., New York, N.Y., all of which are herein incorporated in their entireties by reference for all purposes.

### A. DEFINITIONS

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.” As used herein, the singular forms “a,” “an,” and “the” include the plural reference unless the context clearly dictates otherwise.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Reference to “about” a value or parameter herein includes (and describes) variations that are directed to that value or parameter per se. For example, description referring to “about X” includes description of “X”. Additionally, use of “about” preceding any series of numbers includes “about” each of the recited numbers in that series. For example, description referring to “about X, Y, or Z” is intended to describe “about X, about Y, or about Z.”

The term “average” as used herein refers to either a mean or a median, or any value used to approximate the mean or the median, unless the context clearly indicates otherwise.

A “subject” as used herein refers to an organism, or a part or component of the organism, to which the provided compositions, methods, kits, devices, and systems can be administered or applied. For example, the subject can be a mammal or a cell, a tissue, an organ, or a part of the mammal. As used herein, “mammal” refers to any of the mammalian class of species, preferably human (including humans, human subjects, or human patients). Subjects and mammals include, but are not limited to, farm animals, sport animals, pets, primates, horses, dogs, cats, and rodents such as mice and rats.

As used herein the term “sample” refers to anything which may contain a target molecule for which analysis is desired, including a biological sample. As used herein, a “biological sample” can refer to any sample obtained from a living or viral (or prion) source or other source of macromolecules and biomolecules, and includes any cell type or tissue of a subject from which nucleic acid, protein and/or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine, sweat, semen, stool, sputum, tears, mucus, amniotic fluid or the like, an effusion, a bone marrow sample, ascitic fluid, pelvic wash fluid, pleural fluid, spinal fluid, lymph, ocular fluid, extract of nasal, throat or genital swab, cell suspension from digested tissue, or extract of fecal material, and tissue and organ samples from humans, animals, e.g., non-human mammals, and plants and processed samples derived therefrom.

The terms “polynucleotide,” “oligonucleotide,” “nucleic acid” and “nucleic acid molecule” are used interchangeably herein to refer to a polymeric form of nucleotides of any length, and comprise ribonucleotides, deoxyribonucleotides, and analogs or mixtures thereof. The terms include triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid,” and “nucleic acid molecule” include polydeoxyribonucleotides (containing 2-deoxy-D-ribose), polyribonucleotides (containing D-ribose), including tRNA, rRNA, hRNA, and mRNA, whether spliced or unspliced, any other type of polynucleotide which is an N- or C-glycoside of a purine or pyrimidine base, and other polymers containing nonnucleotidic backbones, for example, polyamide (e.g., peptide nucleic acids (“PNAs”)) and polymorpholino (commercially available from the Anti-Virals, Inc., Corvallis, OR, as Neugene) polymers, and other synthetic sequence-specific nucleic acid polymers providing that the polymers contain nucleobases in a configuration which allows for base pairing and base stacking, such as is found in DNA and RNA. Thus, these terms include, for example, 3′-deoxy-2′,5′-DNA, oligodeoxyribonucleotide N3′ to P5′ phosphoramidates, 2′-O-alkyl-substituted RNA, hybrids between DNA and RNA or between PNAs and DNA or RNA, and also include known types of modifications, for example, labels, alkylation, “caps,” substitution of one or more of the nucleotides with an analog, inter-nucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including enzymes (e.g. nucleases), toxins, antibodies, signal peptides, poly-L-lysine, etc.), those with intercalators (e.g., acridine, psoralen, etc.), those containing chelates (of, e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. A nucleic acid generally will contain phosphodiester bonds, although in some cases nucleic acid analogs may be included that have alternative backbones such as phosphoramidite, phosphorodithioate, or methylphophoroamidite linkages; or peptide nucleic acid backbones and linkages. Other analog nucleic acids include those with bicyclic structures including locked nucleic acids, positive backbones, non-ionic backbones and non-ribose backbones. Modifications of the ribose-phosphate backbone may be done to increase the stability of the molecules; for example, PNA: DNA hybrids can exhibit higher stability in some environments. The terms “polynucleotide,” “oligonucleotide,” “nucleic acid” and “nucleic acid molecule” can comprise any suitable length, such as at least 5, 6, 7, 8, 9, 10, 20, 30, 40, 50, 100, 200, 300, 400, 500, 1,000, 2,000, 3,000, 4,000, 5,000, 6,000, 7,000, 8,000, 9,000, 10,000, or more nucleotides.

It will be appreciated that, as used herein, the terms “nucleoside” and “nucleotide” include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, e.g., wherein one or more of the hydroxyl groups are replaced with halogen, aliphatic groups, or are functionalized as ethers, amines, or the like. The term “nucleotidic unit” is intended to encompass nucleosides and nucleotides.

The terms “complementary” and “substantially complementary” include the hybridization or base pairing or the formation of a duplex between nucleotides or nucleic acids, for instance, between the two strands of a double-stranded DNA molecule or between an oligonucleotide primer and a primer binding site on a single-stranded nucleic acid. Complementary nucleotides are, generally, A and T (or A and U), or C and G. Two single-stranded RNA or DNA molecules are said to be substantially complementary when the nucleotides of one strand, optimally aligned and compared and with appropriate nucleotide insertions or deletions, pair with at least about 80% of the other strand, usually at least about 90% to about 95%, and even about 98% to about 100%. In one aspect, two complementary sequences of nucleotides are capable of hybridizing, preferably with less than 25%, more preferably with less than 15%, even more preferably with less than 5%, most preferably with no mismatches between opposed nucleotides. Preferably the two molecules will hybridize under conditions of high stringency.

As used herein, for a reference sequence, the reverse complementary sequence is the complementary sequence of the reference sequence in the reverse order. For example, for 5′-ATCG-3′, the complementary sequence is 3′-TAGC-5′, and the reverse-complementary sequence is 5′-CGAT-3′.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.” “Hybridization conditions” typically include salt concentrations of approximately less than 1 M, often less than about 500 mM and may be less than about 200 mM. A “hybridization buffer” includes a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are often performed under stringent conditions, i.e., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tₘ for the specific sequence at a defined ionic strength and pH. The melting temperature Tₘ can be the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation, Tₘ=81.5+0.41 (% G+C), when a nucleic acid is in aqueous solution at 1 M NaCl (see e.g., Anderson, M. L. M. and Young, B. D. 1985, Quantitative filter hybridization. In: Nucleic acid hybridization: a practical approach, 73-111. B. D. Hames and S. J. Higgins (eds.). IRL Press Limited, Oxford.). Other references (e.g., Allawi and SantaLucia, Jr., Biochemistry, 36:10581-94 (1997)) include alternative methods of computation which take structural and environmental, as well as sequence characteristics into account for the calculation of Tₘ.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, EDTA) contains 3 M sodium chloride, 0.2 M sodium phosphate, and 0.025 M EDTA. Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a polymerase, for example, a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically. As used herein, ligations are usually carried out enzymatically to form a phosphodiester linkage between a 5′ carbon terminal nucleotide of one oligonucleotide with a 3′ carbon of another nucleotide.

“Amplification,” as used herein, generally refers to the process of producing multiple copies of a desired sequence. “Multiple copies” means at least 2 copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

“Sequence determination” and the like include determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid.

The term “Sequencing,” “High throughput sequencing,” or “next generation sequencing” includes sequence determination using methods that determine many (typically thousands to billions) of nucleic acid sequences in an intrinsically parallel manner, i.e. where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, CT); sequencing by ligation (for example, as commercialized in the SOLiD™ technology, Life Technologies, Inc., Carlsbad, CA); sequencing by synthesis using modified nucleotides (such as commercialized in TruSeq™ and HiSeq™ technology by Illumina, Inc., San Diego, CA; HeliScope™ by Helicos Biosciences Corporation, Cambridge, MA; and PacBio RS by Pacific Biosciences of California, Inc., Menlo Park, CA), sequencing by ion detection technologies (such as Ion Torrent™ technology, Life Technologies, Carlsbad, CA); sequencing of DNA nanoballs (Complete Genomics, Inc., Mountain View, CA); nanopore-based sequencing technologies (for example, as developed by Oxford Nanopore Technologies, LTD, Oxford, UK), and like highly parallelized sequencing methods.

“SNP” or “single nucleotide polymorphism” may include a genetic variation between individuals; e.g., a single nitrogenous base position in the DNA of organisms that is variable. SNPs are found across the genome; much of the genetic variation between individuals is due to variation at SNP loci, and often this genetic variation results in phenotypic variation between individuals. SNPs for use in the present disclosure and their respective alleles may be derived from any number of sources, such as public databases (U.C. Santa Cruz Human Genome Browser Gateway (genome.ucsc.edu/cgi-bin/hgGateway) or the NCBI dbSNP website (ncbi.nlm.nih gov/SNP/), or may be experimentally determined as described in U.S. Pat. No. 6,969,589; and US Pub. No. 2006/0188875 entitled “Human Genomic Polymorphisms.” Although the use of SNPs is described in some of the embodiments presented herein, it will be understood that other biallelic or multi-allelic genetic markers may also be used. A biallelic genetic marker is one that has two polymorphic forms, or alleles. As mentioned above, for a biallelic genetic marker that is associated with a trait, the allele that is more abundant in the genetic composition of a case group as compared to a control group is termed the “associated allele,” and the other allele may be referred to as the “unassociated allele.” Thus, for each biallelic polymorphism that is associated with a given trait (e.g., a disease or drug response), there is a corresponding associated allele. Other biallelic polymorphisms that may be used with the methods presented herein include, but are not limited to multinucleotide changes, insertions, deletions, and translocations.

It will be further appreciated that references to DNA herein may include genomic DNA, mitochondrial DNA, episomal DNA, and/or derivatives of DNA such as amplicons, RNA transcripts, cDNA, DNA analogs, etc. The polymorphic loci that are screened in an association study may be in a diploid or a haploid state and, ideally, would be from sites across the genome. Sequencing technologies are available for SNP sequencing, such as the BeadArray platform (GOLDENGATE™ assay) (Illumina, Inc., San Diego, CA) (see Fan, et al., Cold Spring Symp. Quant. Biol., 68:69-78 (2003)), may be employed.

In some embodiments, the term “methylation state” or “methylation status” refers to the presence or absence of 5-methylcytosine (“5-mC” or “5-mCyt”) at one or a plurality of CpG dinucleotides within a DNA sequence. Methylation states at one or more particular CpG methylation sites (each having two CpG dinucleotide sequences) within a DNA sequence include “unmethylated,” “fully-methylated,” and “hemi-methylated.” The term “hemi-methylation” or “hemimethylation” refers to the methylation state of a double stranded DNA wherein only one strand thereof is methylated. The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample. The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

“Multiplexing” or “multiplex assay” herein may refer to an assay or other analytical method in which the presence, amount and/or methylation state (or methylation status) of multiple targets, e.g., multiple nucleic acid sequences, can be assayed simultaneously by using more than one markers, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

As used herein, “disease or disorder” refers to a pathological condition in an organism resulting from, e.g., infection or genetic defect, and characterized by identifiable symptoms.

### B. PANELS OF ISOLATED POLYNUCLEOTIDES AND RELATED COMPOSITIONS

In one aspect, provided herein is a panel of isolated polynucleotides comprising, consisting of, or consisting essentially of one or at least two isolated polynucleotides, each of said isolated polynucleotides having a polynucleotide sequence of any of Target 1 to Target 83 listed in Table 1, or a complementary or substantially complementary sequence thereof.

TABLE 1
Target Regions

| Target | Genomic Coordinates | Genes | Primer Sequence |
|---|---|---|---|
| Target1 | chr12:25101926-25102126 | LRMP, BCAT1 | TTTTGGTTGTGTGGATTGGGTTTGTGG |
| | | | (SEQ ID NO: 1) |
| Target2 | chr7:50343820-50344020 | IKZF1 | CAAAAACAAAACAACACACCCTCTCCA |
| | | | (SEQ ID NO: 2) |
| Target3 | chr1:1244987-1245214 | ACAP3, PUSL1 | AGTGTGGTTTTGATAGCGGGGAGGGGG |
| | | | (SEQ ID NO: 3) |
| Target4 | chr1:2705949-2706176 | ACTRT2, MMEL1 | GCGGGAGGAGGTTCGTGGGATGTTGTA |
| | | | (SEQ ID NO: 4) |
| Target5 | chr1:156611872-156612099 | BCAN | AAAGGGGGTTTTGTGTTGCGTCGGGAG |
| | | | (SEQ ID NO: 5) |
| Target6 | chr1:237205513-237205740 | RYR2 | TTTTGTTGTCGGGAGTCGGGGGTTGGA |
| | | | (SEQ ID NO: 6) |
| Target7 | chr1:237205892-237206119 | RYR2 | GTTGTTAGGGGAAGGGGGCGTTAGGGT |
| | | | (SEQ ID NO: 7) |
| Target8 | chr10:28034626-28034853 | MKX | GGGGTTGTATTTGTGGTTGCGGGGTCG |
| | | | (SEQ ID NO: 8) |
| Target9 | chr10:31446731-31446958 | ZEB1, ZNF438 | AGGGAGATTTGGGGGGTTGGATGTGTT |
| | | | (SEQ ID NO: 9) |
| Target10 | chr10:74069310-74069537 | DDIT4, DNAJB12 | GGAAGCGGGAGGTATTGGGGGTGTTGA |
| | | | (SEQ ID NO: 10) |
| Target11 | chr10:130084908-130085135 | MKI67 | ATGGGGTGGGCGTTAGTGTAGGTTGGG |
| | | | (SEQ ID NO: 11) |
| Target12 | chr10:133110769-133110996 | PPP2R2D | GTGGATTCGGGATGGGGAGGAGGTTGG |
| | | | (SEQ ID NO: 12) |
| Target13 | chr11:420432-420659 | SIGIRR, ANO9 | ATATGAGTGCGGGGCGTCGGTGAGTTT |
| | | | (SEQ ID NO: 13) |
| Target14 | chr11:73371717-73371944 | PLEKHB1, RAB6A | AGGTTTGTGTTAGGCGTGGGGGTAGGG |
| | | | (SEQ ID NO: 14) |
| Target15 | chr11:125036375-125036602 | PKNOX2, FEZ1 | GGTAGTTTCGCGGGGTTGGGAGTGGAT |
| | | | (SEQ ID NO: 15) |
| Target16 | chr12:4918802-4919029 | KCNA6 | GGTTGGGTTTGGGGAGGTGATTTGGGC |
| | | | (SEQ ID NO: 16) |
| Target17 | chr12:30354393-30354620 | TMTC1, IPO8 | GTGGGGACGTTGAGCGGTCGTTAGGAT |
| | | | (SEQ ID NO: 17) |
| Target18 | chr12:33592747-33592974 | SYT10 | TATGGAGTGTTGAGGGGAGGGGGGAGT |
| | | | (SEQ ID NO: 18) |
| Target19 | chr12:108169315-108169542 | WSCD2, ASCL4 | GGGGGTTTTGGTTAGTTCGGGCGTTCG |
| | | | (SEQ ID NO: 19) |
| Target20 | chr13:25320013-25320240 | RNF17, ATP12A | CGGTTTGTAGGGTGAGTTACGCGGGGA |
| | | | (SEQ ID NO: 20) |
| Target21 | chr13:25320071-25320298 | RNF17, ATP12A | GGCGTAGGAAGGGGAGGTTTCGGGAAG |
| | | | (SEQ ID NO: 21) |
| Target22 | chr13:36049697-36049924 | MAB21L1, NBEA | GGGGATGTGGGGAAGTGGTTAGTGGGT |
| | | | (SEQ ID NO: 22) |
| Target23 | chr13:37006101-37006328 | CCNA1 | TAAAGTCGGGGTTATCGTGGGGTGGGC |
| | | | (SEQ ID NO: 23) |
| Target24 | chr13:51417574-51417801 | RNASEH2B, DLEU1 | GGGTCGTTAGCGGAGGCGATGGTTAGT |
| | | | (SEQ ID NO: 24) |
| Target25 | chr13:109147964-109148191 | MYO16, TNFSF13B | GCGGGTGAGAGGGGTCGGTAGTTGTTT |
| | | | (SEQ ID NO: 25) |
| Target26 | chr13:112758727-112758954 | SPACA7, SOX1 | TAGGTCGGGGTTTAGGGCGTTGGGTTG |
| | | | (SEQ ID NO: 26) |
| Target27 | chr14:48144205-48144432 | ENSG00000272781 | AGTAGGGTTGGGGGTTTCGGATTGCGG |
| | | | (SEQ ID NO: 27) |
| Target28 | chr14:57265370-57265598 | OTX2, TMEM260 | TGGGGGTTTTCGGGGTAGAGATGTGAGT |
| | | | (SEQ ID NO: 28) |
| Target29 | chr14:104710923-104711152 | C14orf144 | TGGAATTTGGGTTTATGGGGGATTCGGGT |
| | | | (SEQ ID NO: 29) |
| Target30 | chr15:68114350-68114577 | PIAS1, SKOR1 | TAATGGTAGGGATGGTTTGGGGCGCGA |
| | | | (SEQ ID NO: 30) |
| Target31 | chr15:96909441-96909668 | SPATA8, NR2F2 | GGGGGGCGGGTTAAAGTGAGAATGGGA |
| | | | (SEQ ID NO: 31) |
| Target32 | chr16:22825689-22825916 | HS3ST2 | GTCGGGGGTTATGGTTTTCGAGGGGGT |
| | | | (SEQ ID NO: 32) |
| Target33 | chr16:51190030-51190257 | SALL1 | GGTGGTAAGCGGAGGGTGGGTTTGGTA |
| | | | (SEQ ID NO: 33) |
| Target34 | chr16:70771598-70771825 | MTSS1L, VAC14 | GGAGGGAGGTCGGAGTGGAGTAGGGAT |
| | | | (SEQ ID NO: 34) |
| Target35 | chr17:75369548-75369775 | TNRC6C, SEPT9 | GGGAGGGCGTTTGGATTAGGGGTTTCG |
| | | | (SEQ ID NO: 35) |
| Target36 | chr17:75369629-75369856 | TNRC6C, SEPT9 | TCGAAGGTGGGTGTTGGGTTGGTTGTT |
| | | | (SEQ ID NO: 36) |
| Target37 | chr19:660388-660615 | RNF126, FGF22 | TTTGGCGTTTGTTTTGGGGAGGGACGG |
| | | | (SEQ ID NO: 37) |
| Target38 | chr19:10404736-10404963 | ICAM5, ZGLP1 | GAGTCGTGGCGGTTGGTGGCGTTGTAG |
| | | | (SEQ ID NO: 38) |
| Target39 | chr19:16511916-16512143 | EPS15L1, KLF2 | AAAGTTGATGATGACGGGTTCGGGGGC |
| | | | (SEQ ID NO: 39) |
| Target40 | chr2:10444970-10445197 | HPCAL1, ODC1 | AATGGAGCGTCGGTATGGGGTTGGGTT |
| | | | (SEQ ID NO: 40) |
| Target41 | chr2:31805739-31805966 | XDH, MEMO1 | GTTCGAAGAGGGAGAGGGGTTGTCGGG |
| | | | (SEQ ID NO: 41) |
| Target42 | chr2:45028929-45029156 | SIX3, CAMKMT | GGAAAGGGCGGTGGTTGTGGGTAAAGC |
| | | | (SEQ ID NO: 42) |
| Target43 | chr2:177017143-177017370 | HOXD1, HOXD4 | GGTGTGTTTGAGGAGGTGGGTGGGAGT |
| | | | (SEQ ID NO: 43) |
| Target44 | chr2:177017395-177017622 | HOXD1, HOXD4 | TGTTGGTTGGGGGCGATTGAGGAGGAG |
| | | | (SEQ ID NO: 44) |
| Target45 | chr20:43331782-43332009 | ADA, WISP2 | TTAGGGGAGTTGATGGGGTTGGTGGCG |
| | | | (SEQ ID NO: 45) |
| Target46 | chr21:38068453-38068680 | SIM2 | TGCGGGTGGGGACGTATTGAGAGGTTC |
| | | | (SEQ ID NO: 46) |
| Target47 | chr21:38068692-38068919 | SIM2 | GTTGGAGGGGGAGTTTCGGGTTTTCGG |
| | | | (SEQ ID NO: 47) |
| Target48 | chr21:47518780-47519007 | COL6A2 | GTTTTGGGATTTCGTCGGGGCGTTGGT |
| | | | (SEQ ID NO: 48) |
| Target49 | chr22:41445010-41445237 | EP300, RBX1 | AGCGGAAGTAGCGTGGATTTGTAGGGG |
| | | | (SEQ ID NO: 49) |
| Target50 | chr22:46403911-46404138 | PPARA, WNT7B | TTTGGGAGTGGGAGTGGGGAGAGTTGC |
| | | | (SEQ ID NO: 50) |
| Target51 | chr3:13323436-13323664 | IQSEC1, NUP210 | GGGCGTTCGAAGGTTTATACGGTGAGGT |
| | | | (SEQ ID NO: 51) |
| Target52 | chr4:24801787-24802014 | SOD3, LGI2 | ATTTTTGGGTGGAGAGGGTTGCGGGGA |
| | | | (SEQ ID NO: 52) |
| Target53 | chr4:44449530-44449757 | KCTD8 | GTTTAGTAGGGGGCGAGGGGTGTTGGG |
| | | | (SEQ ID NO: 53) |
| Target54 | chr4:147559993-147560220 | POU4F2 | CGGGTTAGTCGGGGTTGGAGTTGGGAA |
| | | | (SEQ ID NO: 54) |
| Target55 | chr4:154709572-154709799 | SFRP2 | ATGTCGGGTTAGGGGAAGTCGAAGGCG |
| | | | (SEQ ID NO: 55) |
| Target56 | chr5:1140002-1140229 | SLC6A19, SLC12A7 | TGGGGTGTTGGTTGAGGTTAAGGCGGT |
| | | | (SEQ ID NO: 56) |
| Target57 | chr5:3606411-3606638 | IRX1 | CGGGAGGGAGTAAGGGATGGAGTTGCG |
| | | | (SEQ ID NO: 57) |
| Target58 | chr5:76249564-76249791 | AGGF1, CRHBP | AGGTGGGATTTTGTGCGGGGGGTAGAG |
| | | | (SEQ ID NO: 58) |
| Target59 | chr5:115265894-115266121 | ATG12, | AAGAGATCGACGGGGTGAGTGGTGTGG |
| | | ENSG00000172901 | (SEQ ID NO: 59) |
| Target60 | chr6:6724503-6724734 | RREB1, LY86 | CGGAGGTTGTTAGTGGAAAGTTTGAGGGGGG |
| | | | (SEQ ID NO: 60) |
| Target61 | chr6:41606328-41606555 | MDFI | GGGTTGCGTTGGGGGTTTTATAGGGCG |
| | | | (SEQ ID NO: 61) |
| Target62 | chr6:166970625-166970852 | MPC1, RPS6KA2 | GGGGGACGTGGTGGTTAGGATCGTGTT |
| | | | (SEQ ID NO: 62) |
| Target63 | chr7:70111531-70111760 | WBSCR17 | GCGGGTTTTGAGTAGTCGTTTTGTATGGT |
| | | | (SEQ ID NO: 63) |
| Target64 | chr7:70596236-70596463 | WBSCR17 | GGGATCGCGCGGGATGGAGGTAGTTAA |
| | | | (SEQ ID NO: 64) |
| Target65 | chr7:97361672-97361899 | TAC1 | TGAAGGTGTGGGTTGGTGGGTTAGGGG |
| | | | (SEQ ID NO: 65) |
| Target66 | chr7:152622494-152622721 | ACTR3B | GTTTGGTTCGGTTTTGGCGAAGGGGGT |
| | | | (SEQ ID NO: 66) |
| Target67 | chr7:155167519-155167746 | EN2, INSIG1 | AAAGGCGGATTGAGGGTGGAGGAGGTC |
| | | | (SEQ ID NO: 67) |
| Target68 | chr7:157481309-157481536 | DNAJB6, PTPRN2 | CGGGGAAGCGGGGGAGAAAGTTTAGGT |
| | | | (SEQ ID NO: 68) |
| Target69 | chr8:23564079-23564306 | NKX2-6 | GAAGGGGGTGGAGGTGACGGGGTTTAG |
| | | | (SEQ ID NO: 69) |
| Target70 | chr8:55382643-55382870 | RP1, SOX17 | TAGGGCGTGGGTAGTGGTGGTAGTGGA |
| | | | (SEQ ID NO: 70) |
| Target71 | chr8:70946907-70947134 | SLCO5A1, PRDM14 | AGAGAGGTATCGGGCGAGAAGGGGTGT |
| | | | (SEQ ID NO: 71) |
| Target72 | chr8:97171974-97172201 | GDF6 | AGGCGGATGAATTCGGTTGGAGAGGGG |
| | | | (SEQ ID NO: 72) |
| Target73 | chr8:97506313-97506540 | SDC2 | GGGAGTAGGCGTAGGAGGAGGAAGCGA |
| | | | (SEQ ID NO: 73) |
| Target74 | chr9:19788555-19788782 | SLC24A2 | GGTGGTTTTGTCGTGGATGTGGGCGTT |
| | | | (SEQ ID NO: 74) |
| Target75 | chr9:19789006-19789233 | SLC24A2 | CGTTAGTCGGGTTGGGTTCGGGAGGAG |
| | | | (SEQ ID NO: 75) |
| Target76 | chr9:68413040-68413267 | ANKRD20A1, | GATGGTGAGCGAAGAGGCGGAGGAGTT |
| | | FOXD4L6 | (SEQ ID NO: 76) |
| Target77 | chr10:131771113-131771340 | GLRX3, EBF3 | GAAGGTAGTGTGGCGCGTGCGTATTGG |
| | | | (SEQ ID NO: 77) |
| Target78 | chr14:57274926-57275153 | OTX2, TMEM260 | GAGGTTAGCGTCGGTCGTTTTTCGCGG |
| | | | (SEQ ID NO: 78) |
| Target79 | chr16:58497281-58497508 | NDRG4 | GCGGTGTCGGGTTTGGGGTTAGTTTCG |
| | | | (SEQ ID NO: 79) |
| Target80 | chr3:192125834-192126061 | FGF12 | TCGGAGGGCGTAGTAGAGGTTGGGGTT |
| | | | (SEQ ID NO: 80) |
| Target81 | chr4:8859815-8860042 | HMX1, CPZ | GGGGGAGGGGGTAGTAGGTCGATCGTT |
| | | | (SEQ ID NO: 81) |
| Target82 | chr5:16180191-16180418 | MARCH11 | GGGAAGTAGGGGGTAGGGGAGAGGTCG |
| | | | (SEQ ID NO: 82) |
| Target83 | chr8:70981894-70982121 | SLCO5A1, PRDM14 | GGGGTATGGCGGGGGTAGTAGACGTTG |
| | | | (SEQ ID NO: 83) |

The present panel can comprise, consist of, or consist essentially of any suitable number of the above isolated polynucleotides. For example, the present panel can comprise, consist of, or consist essentially of one isolated polynucleotide, said isolated polynucleotides having a polynucleotide sequence of any of Target 1 to Target 83 listed in Table 1, or a complementary or substantially complementary sequence thereof. In another example, the present panel can comprise, consist of, or consist essentially of at least 2, 3, 4, 5, 6, 7, 8, 9, 10, 20, 30, 40, 50, 60, 70, 80, or 83 of the isolated polynucleotides, or the isolated polynucleotides corresponding to all of Target 1 to Target 83 listed in Table 1.

In one embodiment, the present panel can comprise at least one or at least 2, 3, 4, 5, 6, 7, 8, 9, 10, 20, 30, 40, 50, 60, 70, 80, or 83 of the isolated polynucleotides, or the isolated polynucleotides corresponding to all of Target 1 to Target 83 listed in Table 1. In another embodiment, the present panel can consist of at least one or at least 2, 3, 4, 5, 6, 7, 8, 9, 10, 20, 30, 40, 50, 60, 70, 80, or 83 of the isolated polynucleotides, or the isolated polynucleotides corresponding to all of Target 1 to Target 83 listed in Table 1. In still another embodiment, the present panel can consist essentially of at least one or at least 2, 3, 4, 5, 6, 7, 8, 9, 10, 20, 30, 40, 50, 60, 70, 80, or 83 of the isolated polynucleotides, or the isolated polynucleotides corresponding to all of Target 1 to Target 83 listed in Table 1.

The isolated polynucleotides in the present panel can be any suitable type(s) of polynucleotides. For example, the isolated polynucleotides can be DNA molecules, RNA molecules, or a combination thereof. In some embodiments, the DNA molecules can be genomic DNA molecules or fragments thereof.

The isolated polynucleotides can be immobilized on a substrate. The isolated polynucleotides can be immobilized on any suitable substrate. For example, the substrate can comprise a solid surface, a porous surface, or a combination thereof. In some embodiments, the substrate can be a part of a bead, a tube, a microtiter plate, a membrane, a gel, or a glass slide. In other embodiments, the isolated polynucleotide molecules can be immobilized spatially apart from each other on a substrate so that each of the isolated polynucleotide molecules can be assessed or analyzed individually.

Kits, devices, systems or articles of manufacture that comprise any of the above panel(s) are also provided.

The present kits, devices, systems or articles of manufacture can be configured for any suitable use or purpose. For example, the present kits, devices, systems or articles of manufacture can be configured for assessing methylation status of one or at least two of Target 1 to Target 83 listed in Table 1, e.g., configured for in vitro assessing of methylation status of one or at least two of Target 1 to Target 83 listed in Table 1 in one or more blood or plasma sample(s) obtained from one or more subject(s). In some embodiments, the present kits, devices, systems or articles of manufacture can be configured for assessing methylation status of at least 2, 3, 4, 5, 6, 7, 8, 9, 10, 20, 30, 40, 50, 60, 70, 80, or 83 of Target 1 to Target 83 listed in Table 1, or the isolated polynucleotides corresponding to all of Target 1 to Target 83 listed in Table 1, or a numerical range or subrange thereof. In other embodiments, the isolated polynucleotides in the present kits, devices, systems or articles of manufacture can be configured as control polynucleotides.

The isolated polynucleotides in the present kits, devices, systems or articles of manufacture can have any suitable level of concentration. For example, the isolated polynucleotides can have a level of concentration from about 1 femtomolar to about 100 millimolar, e.g., at about 1 femtomolar (fM), 10 fM, 100 fM, 1 picomolar (pM), 10 μM, 100 μM, 1 nanomolar (nM), 10 nM, 100 nM, 1 micromolar (μM), 10 μM, 100 μM, 1 millimolar (mM), 10 mM, 100 mM, or a subrange thereof.

The present kits, devices, systems or articles of manufacture can be configured for any suitable use or purpose. For example, the kits, devices, systems or articles of manufacture can be configured for assessing colorectal cancer or advanced adenoma in a subject.

### C. KITS, SYSTEMS AND RELATED COMPOSITIONS FOR ASSESSING METHYLATION

In another aspect, a kit, device, system or an article of manufacture that is configured for assessing methylation status of one or at least two of Target 1 to Target 83 listed in Table 1 is provided. In some embodiments, the present kits, devices, systems or articles of manufacture are configured for in vitro assessing of methylation status of one or at least two of Target 1 to Target 83 listed in Table 1 in one or more blood or plasma sample(s) obtained from one or more subject(s).

The present kits, devices, systems or articles of manufacture can comprise reagents for assessing methylation status of any suitable number of the Targets listed in Table 1. For example, the present kits, devices, systems or articles of manufacture can comprise reagents for assessing methylation status of at least 1, 2, 3, 4, 5, 6, 7, 8, 9, 10, 20, 30, 40, 50, 60, 70, 80, or 83 of the isolated polynucleotides, or the isolated polynucleotides corresponding to all of Target 1 to Target 83 listed in Table 1.

In some embodiments, the present kits, devices, systems or articles of manufacture can comprise reagents for assessing methylation status of any subgroup of the Targets listed in Table 1. For example, the present kits, devices, systems or articles of manufacture can comprise reagents for assessing methylation status of Targets 1-10, 11-20, 21-30, 31-40, 41-50, 51-60, 61-70, 71-80, etc., listed in Table 1. In other embodiments, the present kits, devices, systems or articles of manufacture can comprise reagents for assessing methylation status of all of the Targets listed in Table 1, excluding one or more Targets listed in Table 1. For example, the present kits, devices, systems or articles of manufacture can comprise reagents for assessing methylation status of Targets 1-82 listed in Table 1, but do not comprise reagent(s) for assessing methylation status of Target 83 listed in Table 1, etc.

The present kits, devices, systems or articles of manufacture can comprise any suitable reagents for assessing methylation status of the target polynucleotides. For example, the reagents can comprise, consist essentially of, or consist of a probe or primer configured for hybridizing with each of the targets whose methylation status is to be assessed. In some embodiments, the reagents comprise, consist essentially of, or consist of a single probe or primer configured for hybridizing with each of the targets whose methylation status is to be assessed. In other embodiments, the reagents comprise, consist essentially of, or consist of multiple probes or primers configured for hybridizing with each of the targets whose methylation status is to be assessed

In some embodiments, the one or more primers in the present kits, devices, systems or articles of manufacture comprise, consist essentially of, or consist of a sequence set forth in SEQ ID NOs: 1-83 listed in Table 1, a complementary or substantially complementary sequence thereof, or any combination thereof. For each Target listed in Table 1, the present kits, devices, systems or articles of manufacture can comprise, consist essentially of, or consist of one or more corresponding primers for that Target. For example, the one or more primers may comprise, consist essentially of, or consist of a sequence set forth in any of SEQ ID NO:1-SEQ ID NO: 83, a complementary or substantially complementary sequence thereof, or any combination thereof.

In some embodiments, the present kits, devices, systems or articles of manufacture can comprise a common primer, e.g., a common primer for amplifying each of the targets whose methylation status is to be assessed. An exemplary common primer can comprise, consist essentially of, or consist of a sequence set forth in SEQ ID NO:84 (CACTCTTTCCCTACACGACGC), or a complementary or substantially complementary sequence thereof.

The present kits, devices, systems or articles of manufacture can further comprise any other suitable reagents. In some embodiments, the present kits, devices, systems or articles of manufacture can further comprise any other suitable reagents disclosed and/or claimed in U.S. provisional application Ser. No. 62/487,422, filed on Apr. 19, 2017, U.S. provisional application Ser. No. 62/487,423, filed on Apr. 19, 2017, US 2020/0048697 A1 and US 2020/0123538 A1.

For example, the present kits, devices, systems or articles of manufacture can further comprise an agent for isolating the targets from a sample, e.g., a blood or plasma sample.

In another example, the present kits, devices, systems or articles of manufacture can further comprise a reagent for preparing a library of the targets. Any suitable reagents for preparing a library of the targets can be included. In some embodiments, the reagent for preparing a library of the targets can comprise an enzyme, e.g., a ligase or a single-stranded DNA (ssDNA) ligase. Any suitable ssDNA ligase can be included, e.g., a Thermus bacteriophage RNA ligase such as a bacteriophage TS2126 RNA ligase (e.g., CircLigase™ and CircLigase II™), or an archaebacterium RNA ligase such as Methanobacterium thermoautotrophicum RNA ligase 1.

In still another example, the present kits, devices, systems or articles of manufacture can further comprise a reagent for amplifying the targets or a library of the targets. Any suitable reagents for amplifying the targets or a library of the targets can be included. In some embodiments, the reagent for amplifying the targets or a library of the targets can comprise an enzyme, e.g., an enzyme to be used in a polynucleotide amplification reaction. Exemplary polynucleotide amplification reactions include polymerase chain reaction (PCR), strand displacement amplification (SDA), transcription mediated amplification (TMA), ligase chain reaction (LCR), nucleic acid sequence based amplification (NASBA), primer extension, rolling circle amplification (RCA), self sustained sequence replication (3SR), and loop-mediated isothermal amplification (LAMP).

In yet another example, the present kits, devices, systems or articles of manufacture can further comprise an agent for purifying the targets, a library of the targets, amplified targets or a library of amplified targets,

In yet another example, the present kits, devices, systems or articles of manufacture can further comprise a reagent for assessing methylation status of the targets. Any suitable reagents for assessing methylation status of the targets can be included. In some embodiments, the reagent for assessing methylation status of the targets can be a reagent to be used in a polynucleotide methylation, e.g., DNA methylation, detecting method. Exemplary polynucleotide methylation or DNA methylation detecting methods include mass spectrometry, methylation-specific PCR (MSP), bisulphite sequencing, the HpaII tiny fragment Enrichment by ligation-mediated PCR assay (HELP Assay), Glal hydrolysis and ligation adapter dependent PCR assay (GLAD-PCR assay), restriction landmark genomic scanning (RLGS), methylated DNA immunoprecipitation (MeDIP or mDIP), pyrosequencing, molecular break light assay for DNA adenine methyltransferase activity, methyl sensitive Southern blotting and high resolution Melt (HRM) analysis.

The reagent for assessing methylation status of the targets can be a chemical agent, e.g., bisulfite or sodium bisulfite. The reagent for assessing methylation status of the targets can also be a biological agent, e.g., a polypeptide or an enzyme.

Any suitable enzyme can be included. In some embodiments, the enzyme can be a methylation-sensitive restriction enzyme (MSRE). The MSRE can selectively cleave at a residue when it is unmethylated. The MSRE can also selectively cleave at the residue when it is methylated. Exemplary MSRE can be selected from the group consisting of HpaII, SalI, SalI-HF®, ScrFI, BbeI, NotI, SmaI, XmaI, MboI, BstBI, ClaI, MluI, NaeI, NarI, PvuI, SacII, HhaI, and a combination thereof.

In some embodiments, the enzyme can be a polynucleotide polymerase. The polynucleotide polymerase is configured to be used in polynucleotide amplification reaction, e.g., PCR. Any suitable polynucleotide polymerase can be included. For example, the polynucleotide polymerase can be a DNA polymerase, e.g., a DNA polymerase without a 3′ to 5′ exonuclease activity.

In yet another example, the present kits, devices, systems or articles of manufacture can further comprise a denaturing reagent for denaturing a double-stranded polynucleotide from a sample to obtain the single-stranded polynucleotide.

In yet another example, the present kits, devices, systems or articles of manufacture can further comprise a crowding agent for the ligation reaction. In one aspect, the crowding agent comprises a polyethylene glycol (PEG), such as PEG 4000 or PEG 6000, Dextran, and/or Ficoll.

In yet another example, the present kits, devices, systems or articles of manufacture can further comprise a set of primers each comprising a sequence that is reverse-complement to the adaptor and/or hybridizable to the adaptor, for converting the single-stranded polynucleotide to a double-stranded polynucleotide.

In yet another example, the present kits, devices, systems or articles of manufacture can further comprise a reagent for removing primer dimer and/or primer-adaptor duplex.

In yet another example, the present kits, devices, systems or articles of manufacture can further comprise a primer comprising a sequence specific for a target sequence (e.g., a target sequence listed in Table 1), for obtaining an amplified linear, double-stranded ligation product comprising sequence information of the target sequence. In a further aspect, the kit can further comprise a sequencing adapter and/or a sample-specific barcode, for sequencing the amplified linear, double-stranded ligation product.

In yet another example, the present kits, devices, systems or articles of manufacture can further comprise a panel of isolated polynucleotides described in the above Section B.

In yet another example, the present kits, devices, systems or articles of manufacture can further comprise a reference sample and/or information of a control locus.

In yet another example, the present kits, devices, systems or articles of manufacture can further comprise separate containers, e.g., vials, for one or more components and/or instructions for using the kits, devices, systems or articles of manufacture.

In yet another example, the present kits, devices, systems or articles of manufacture can further comprise a computer readable medium containing executable instructions for obtaining a methylation metric of a sample based on the methylation status assessment, The computer readable medium can be configured for obtaining a methylation metric in any suitable form, e.g., in the form of average methylation frequency, methylation haplotype load, unmethylation haplotype load, percent discordant reads, or a combination thereof.

In yet another example, the present kits, devices, systems or articles of manufacture can further comprise a computer readable medium containing executable instructions for classification using the methylation metric(s). Exemplary classification algorithm can be linear discriminant analysis, logistic regression, naïve bayes classification, perceptron classification, quadratic classification, k-nearest neighbors, boosting, decision tree, random forest, neural network, learning vector quantization, or support vector machines, or a combination thereof.

The present kits, devices, systems or articles of manufacture can be configured for any suitable use or purpose. For example, the present kits, devices, systems or articles of manufacture can be configured for assessing colorectal cancer or advanced adenoma in a subject.

In some embodiments, the kit may additionally comprise reagents for detecting presence of polypeptides. Such reagents may be antibodies or other binding molecules that specifically bind to a polypeptide. In some embodiments, such antibodies or binding molecules may be capable of distinguishing a structural variation to the polypeptide as a result of polymorphism, and thus may be used for genotyping. The antibodies or binding molecules may be labeled with a detectable marker, such as, for example, a radioisotope, fluorescent compound, bioluminescent compound, a chemiluminescent compound, metal chelator or enzyme. Other reagents for performing binding assays, such as ELISA, may be included in the kit.

In some embodiments, the kits comprise reagents for genotyping at least two, at least three, at least five, at least ten, or more markers. The markers may be a polynucleotide marker (such as a cancer-associated mutation or SNP) or a polypeptide marker (such as overexpression or a post-translational modification, including hyper- or hypo-phosphorylation, of a protein) or any combination thereof. In some embodiments, the kits may further comprise a surface or substrate (such as a microarray) for capture probes for detecting of amplified nucleic acids.

The kits may further comprise a carrier means being compartmentalized to receive in close confinement one or more container means such as vials, tubes, and the like, each of the container means comprising one of the separate elements to be used in the method. For example, one of the container means may comprise a probe that is or can be detectably labeled. Such probe may be a polynucleotide specific for a biomarker. The kit may also have containers containing nucleotide(s) for amplification of the target nucleic acid sequence and/or a container comprising a reporter-means bound to a reporter molecule, such as an enzymatic, florescent, or radioisotope label.

The kit typically comprises the container(s) described above and one or more other containers comprising materials desirable from a commercial and user standpoint, including buffers, diluents, filters, needles, syringes, and package inserts with instructions for use. A label may be present on the container to indicate that the composition is used for a specific therapy or non-therapeutic application, and may also indicate directions for either in vivo or in vitro use, such as those described above.

The kit can further comprise a set of instructions and materials for preparing a tissue or cell or body fluid sample and preparing nucleic acids (such as ctDNA) from the sample, e.g., a blood sample, a serum sample or a plasma sample.

### D. METHODS FOR ASSESSING CANCER OR NEOPLASIA IN A SUBJECT

In still another aspect, provided herein is an in vitro method for classifying a blood sample, which method comprises: a) in vitro assessing methylation status of one or at least two target polynucleotides in a blood sample from a subject, each of said target polynucleotides having a polynucleotide sequence of any of Target 1 to Target 83 listed in Table 1, or a complementary or substantially complementary sequence thereof; and b) classifying said blood sample into a category based on said methylation status of said one or at least two target polynucleotides in said blood sample obtained in a).

The present methods can be used for any suitable purposes. In some embodiments, step b) of the present methods can comprise generating a methylation status score with a computer algorithm based on the assessed methylation status of the one or at least two target polynucleotides, and classifying the blood sample into a category based on the methylation status score. In some embodiments, the present methods can be used for assessing colorectal cancer and/or advanced adenoma in a subject.

The present methods can be used on any suitable blood sample, e.g., any suitable sample obtained or derived from a subject whose colorectal cancer and/or advanced adenoma status is to be assessed. For example, the blood sample can comprise circulating cell-free or tumor DNA (ctDNA). In another example, the blood sample can be a whole blood sample, a serum sample, a plasma sample, or any combination thereof.

The target or template polynucleotides can be target DNA molecules, RNA molecules, or a complex or a combination thereof. DNA can include be regular genomic DNA, chromosomal DNA, extrachromosomal DNA (such as mitochondrial DNA), or a fragment thereof. In other embodiments, the target or template DNA is a processed DNA, for example, one that has undergone enzyme digestion, cross-linking, chemical or physical shearing, bisulfite conversion, and/or degradation.

In some embodiments, the target nucleic acid molecule of interest disclosed herein is a cell-free DNA, such as cell-free fetal DNA (also referred to as “cfDNA”) or ctDNA. cfDNA circulates in the body, such as in the blood, of a pregnant mother, and represents the fetal genome, while ctDNA circulates in the body, such as in the blood, of a cancer patient, and is generally pre-fragmented. In other embodiments, the target nucleic acid molecule of interest disclosed herein is an ancient and/or damaged DNA, for example, due to storage under damaging conditions such as in formalin-fixed samples, or partially digested samples.

As cancer cells die, they release DNA into the bloodstream. This DNA, known as circulating tumor DNA (ctDNA), is highly fragmented, with an average length of approximately 150 base pairs. Once the white blood cells are removed, ctDNA generally comprises a very small fraction of the remaining plasma DNA, for example, ctDNA may constitute less than about 10% of the plasma DNA. Generally, this percentage is less than about 1%, for example, less than about 0.5% or less than about 0.01%. Additionally, the total amount of plasma DNA is generally very low, for example, at about 10 ng/ml of plasma.

The variants in the ctDNA can be interrogated using various methods, including next generation sequencing. Due to the low ratio of ctDNA to plasma DNA, it is difficult to call a variant with high confidence due to PCR and sequencing errors. Unique molecular identifiers (UMIs) can be used to tag original molecules such that any variant seen can be compared to a consensus sequence. This is an effective manner to separate true from false positives. If the variant is matched to a consensus, it is a true positive. Otherwise, it is removed from analysis. Furthermore, it is essential that a high percentage of original molecules are turned into sequencing libraries so that the sensitivity remains high, i.e., variants are not missed due to dropout. Thus, ligation efficiency can be important during library construction.

In one aspect, a technique to improve ligation efficiency while still targeting selected regions of the genome, as disclosed and/or claimed in U.S. provisional application Ser. No. 62/487,423, filed on Apr. 19, 2017, and US 2020/0123538 A1, can be used to prepare target polynucleotides to be analyzed in the present methods. In one embodiment, polynucleotides to be detected by sequencing, such as ctDNA, are first dephosphorylated to remove 5′ phosphates to prevent ligation of ctDNA to itself. The ctDNA is then denatured such that all DNA is single stranded. Circligase™, a single stranded DNA ligase, is used to ligate an adapter to the 3′ end of the ctDNA. In one aspect, the adapter contains 2 specific bases on the 5′ end to optimize ligation efficiency, followed by a UMI. In one aspect, the 3′ end of the adapter contains a carbon spacer to prevent self-ligation of the adapters. In another aspect, the ligation reaction is further optimized using a crowding agent, such as PEG 4000. In one aspect, following ligation, molecules are double-stranded using a primer that is reverse complement to the adapter. This allows efficient removal of excess unligated adapters without removed usable DNA by a standard purification.

In one aspect, the DNA is then amplified using a semi-targeted PCR. One primer can be reverse complement to the adapter, while the other (e.g., as one primer in a primer pool) can anneal to specific, targeted regions of the genome. The specific primers are designed to minimize primer-dimer interactions and off-target annealing. In one aspect, the target-specific primers are further optimized to land in close proximity to specific variants due to the small DNA size. Following another cleanup, a PCR adds the full-length sequencing adapters and barcodes. The final library is then sequenced, for example, on an Illumina machine.

In one aspect, the semi-targeted PCR results in enrichments of >about 40,000 fold of the original molecule set despite having a relatively small target region of ~30,000 bp. In one aspect, the overall conversion rates of the present method are at least 60%, implying that at least ~3 times more of the original molecules are converted into sequenceable material when compared to standard library construction and hybridization capture. In other embodiments, the overall conversion rates are between about 60% and about 70%, between about 70% and about 80%, between about 80% and about 90%, or over 90%. In one aspect, the present method thus is able to accurately call genetic or genomic variants, such as SNVs, indels, CNVs, and fusions at extremely low mutant allele fractions, for example, as low as 0.01%. In other aspects, the allele fraction of the genetic or genomic variant is about 0.05%, about 0.1%, about 0.5%, about 1%, or about 2%.

The present methods can be used for assessing methylation status, colorectal cancer and/or advanced adenoma in any suitable subject. For example, the present methods can be used for assessing methylation status, colorectal cancer and/or advanced adenoma in a mammal. The mammal can be a non-human mammal, e.g., a pet, a farm animal, a companion animal or an experimental animal. Preferably, the mammal is a human. For example, a subject can be a human that needs to be screened for colorectal cancer and/or advanced adenoma risks, a human in a high-risk group, a human that has been diagnosed as having colorectal cancer and/or advanced adenoma but needs further stratification, a human that has been diagnosed as having colorectal cancer and/or advanced adenoma and under active treatment, or a human having colorectal cancer and/or advanced adenoma and is in remission.

The present methods can comprise assessing methylation status of any suitable number of the Targets listed in Table 1. For example, the methods can comprise assessing methylation status of at least 1, 2, 3, 4, 5, 6, 7, 8, 9, 10, 20, 30, 40, 50, 60, 70, 80, or 83 of the isolated polynucleotides, or the isolated polynucleotides corresponding to all of Target 1 to Target 83 listed in Table 1, or a numerical range or subrange thereof.

The methylation status of target polynucleotides can be assessed using any suitable methods or reagents. For example, the methylation status of target polynucleotide(s) can be assessed using a probe or primer configured for hybridizing with each of the target polynucleotide(s). In some embodiments, the methylation status of a target polynucleotide can be assessed using a single probe or primer configured for hybridizing with the target polynucleotide. In other embodiments, the methylation status of a target polynucleotide can be assessed using multiple probes or primers configured for hybridizing with the target polynucleotide.

In some embodiments, the one or more primers used in the in the present methods can comprise, consist essentially of, or consist of a sequence set forth in SEQ ID NOs: 1-83 listed in Table 1, a complementary or substantially complementary sequence thereof, or any combination thereof. For each Target listed in Table 1, the one or more primers used in the present methods can comprise, consist essentially of, or consist of one or more corresponding primers for that Target. For example, the one or more primers may comprise, consist essentially of, or consist of a sequence set forth in any of SEQ ID NO:1-SEQ ID NO:83, a complementary or substantially complementary sequence thereof, or any combination thereof.

The present methods can further comprise using a common primer for amplifying each of the target polynucleotide whose methylation status is to be assessed. An exemplary common primer can comprise, consist essentially of, or consist of a sequence set forth in SEQ ID NO: 84 (CACTCTTTCCCTACACGACGC), or a complementary or substantially complementary sequence thereof.

The present methods can further comprise any other suitable steps. In some embodiments, the present methods can further comprise any other suitable steps disclosed and/or claimed in U.S. provisional application Ser. No. 62/487,422, filed on Apr. 19, 2017, U.S. provisional application Ser. No. 62/487,423, filed on Apr. 19, 2017, US 2020/0048697 A1 and US 2020/0123538 A1. For example, techniques and steps for constructing single-stranded polynucleotide, conversion of single-stranded polynucleotide library to double-stranded polynucleotide library, semi-targeted amplification of double-stranded polynucleotide library, and construction of sequence library and analysis of sequencing reads disclosed and/or claimed in U.S. provisional application Ser. No. 62/487,423, filed on Apr. 19, 2017 and US 2020/0123538 A1, can be used to obtain and/or prepare target polynucleotides to be analyzed.

For example, the present methods can further comprise isolating the target polynucleotides from a sample.

In another example, the present methods can further comprise preparing a library of the target polynucleotides. Any suitable reagents for preparing a library of the targets can be used. In some embodiments, the reagent for preparing a library of the targets can comprise an enzyme, e.g., a ligase or a single-stranded DNA (ssDNA) ligase. Any suitable ssDNA ligase can be included, e.g., a Thermus bacteriophage RNA ligase such as a bacteriophage TS2126 RNA ligase (e.g., CircLigase™ and CircLigase II™), or an archaebacterium RNA ligase such as Methanobacterium thermoautotrophicum RNA ligase 1.

In still another example, the present methods can further comprise amplifying the target polynucleotides. Any suitable reagents for amplifying the targets or a library of the targets can be used. In some embodiments, the reagent for amplifying the targets or a library of the targets can comprise an enzyme, e.g., an enzyme to be used in a polynucleotide amplification reaction. Exemplary polynucleotide amplification reactions include polymerase chain reaction (PCR), strand displacement amplification (SDA), transcription mediated amplification (TMA), ligase chain reaction (LCR), nucleic acid sequence based amplification (NASBA), primer extension, rolling circle amplification (RCA), self-sustained sequence replication (3SR), and loop-mediated isothermal amplification (LAMP).

In yet another example, the present methods can further comprise purifying the target polynucleotide(s), a library of the target polynucleotide(s), amplified target polynucleotide(s) or a library of amplified target polynucleotides.

The methylation status of target polynucleotide(s) can be assessed using any suitable methods and/or regents. In some embodiments, the methylation status of target polynucleotide(s) can be assessed using mass spectrometry, methylation-specific PCR (MSP), methylation-sensitive sequencing, e.g., bisulphite sequencing, the HpaII tiny fragment Enrichment by ligation-mediated PCR assay (HELP Assay), Glal hydrolysis and ligation adapter dependent PCR assay (GLAD-PCR assay), restriction landmark genomic scanning (RLGS), methylated DNA immunoprecipitation (MeDIP or mDIP), pyrosequencing, molecular break light assay for DNA adenine methyltransferase activity, methyl sensitive Southern blotting or high resolution Melt (HRM) analysis.

In other embodiments, the methylation status of target polynucleotides can be assessed using a chemical agent, e.g., bisulfite or sodium bisulfite. In still other embodiments, the methylation status of target polynucleotides can be assessed using a biological agent, e.g., a polypeptide or an enzyme.

Any suitable enzyme can be used. In some embodiments, the enzyme can be a methylation-sensitive restriction enzyme (MSRE). The MSRE can selectively cleave at a residue when it is unmethylated. The MSRE can also selectively cleave at the residue when it is methylated. Exemplary MSRE can be selected from the group consisting of HpaII, SalI, SalI-HF®, ScrFI, BbeI, NotI, SmaI, XmaI, MboI, BstBI, ClaI, MluI, NaeI, NarI, PvuI, SacII, HhaI, and a combination thereof.

In some embodiments, the enzyme can be a polynucleotide polymerase. The polynucleotide polymerase can be used in polynucleotide amplification reaction, e.g., PCR. Any suitable polynucleotide polymerase can be used. For example, the polynucleotide polymerase can be a DNA polymerase, e.g., a DNA polymerase without a 3′ to 5′ exonuclease activity.

In some embodiments, the methylation status of target polynucleotides can be assessed using methylation-sensitive sequencing, e.g., bisulphite sequencing. Bisulfite conversion is a method that uses bisulfite to determine the methylation pattern of DNA. DNA methylation is a biochemical process involving the addition of a methyl group to the cytosine or adenine DNA nucleotides. DNA methylation stably alters the expression of genes in cells as cells divide and differentiate from embryonic stem cells into specific tissues. In bisulfite conversion, target nucleic acids are first treated with bisulfite reagents that specifically convert un-methylated cytosines to uracils while having no impact of methylated cytosine. One consequence of bisulfite conversion is that the double-stranded conformation of the original target is disrupted due to loss of sequence complementarity. The target sequences exist as two separate single-stranded DNAs during sample preparation and analytical or diagnostic testing. Target nucleic acid sequences frequently also exist at very low concentrations. This is an especially important consideration for circulating tumor DNA (also referred to as “cell-free tumor DNA,” or “ctDNA”) due to its often low concentration in circulation and the very low variant allele fraction.

Any suitable format of methylation-sensitive sequencing can be used. For example, the methylation-sensitive sequencing can be conducted with a format selected from the group consisting of Maxam-Gilbert sequencing, a chain-termination method, shotgun sequencing, bridge PCR, single-molecule real-time sequencing, ion semiconductor (ion torrent sequencing), sequencing by synthesis, sequencing by ligation (SOLID sequencing), chain termination (Sanger sequencing), massively parallel signature sequencing (MPSS), polony sequencing, 454 pyrosequencing, Illumina (Solexa) sequencing, DNA nanoball sequencing, heliscope single molecule sequencing, single molecule real time (SMRT) sequencing, nanopore DNA sequencing, tunnelling currents DNA sequencing, sequencing by hybridization, sequencing with mass spectrometry, microfluidic Sanger sequencing, a microscopy-based technique, RNAP sequencing, and in vitro virus high-throughput sequencing.

In some embodiments, the present methods can further comprise, prior to the methylation-sensitive sequencing, e.g., bisulphite sequencing, obtaining a library of linear, single-stranded ligation products, each of the linear, single-stranded ligation products comprises of a linear, single-stranded target polynucleotide linked to an adaptor comprising a unique molecular identifier (UMI) sequence that earmarks the single-stranded target polynucleotide to which the adaptor is ligated. The sequencing reads from the target polynucleotides can be first adapter trimmed to remove any adapter sequence originating from the library construction process to obtain trimmed sequencing reads. The trimmed sequencing reads can be mapped to a reference genome, e.g., a human reference genome, using an alignment program to obtain an aligned read file. The aligned read file can be grouped into sets corresponding to each target region from Target 1 to Target 83 listed in Table 1, or a complementary or substantially complementary sequence thereof, that can be used for methylation status assessment.

Methylation status of one, more or each of the target polynucleotides can be assessed. In some embodiments, the methylation status of each of the target polynucleotides can be assessed to obtain a methylation metric, e.g., in the form of average methylation frequency, methylation haplotype load, unmethylation haplotype load, percent discordant reads, or a combination thereof. In some embodiments, methylation status of a blood sample is assessed using the methylation metrics from each of the target polynucleotides.

Methylation status of a sample can be assessed using the methylation metrics from each of the target polynucleotides. The methylation metrics can be analyzed or used in any suitable manner. For example, the methylation metric for each of the target polynucleotides can be compared to a threshold or reference value to assess methylation status of a sample.

In another example, a numerical methylation matrix can be computed using the methylation metrics from each of the target polynucleotides to assess methylation status of a sample. The numerical methylation matrix can be in any suitable format. In some embodiments, the numerical methylation matrix from a sample can comprise a single numerical number or value. In other embodiments, the numerical methylation matrix from a sample can comprise multiple numerical numbers or values. The numerical methylation matrix can be obtained or computed using any suitable formulae or algorithm. In some embodiments, the numerical methylation matrix can be computed using a classification algorithm, e.g., linear discriminant analysis, logistic regression, naïve bayes classification, perceptron classification, quadratic classification, k-nearest neighbors, boosting, decision tree, random forest, neural network, learning vector quantization, or support vector machines, or a combination thereof.

The methylation matrix can be used in any suitable manner in assess methylation status of a sample. For example, the methylation matrix can be compared to a threshold or reference value to assess methylation status of a sample.

The methylation status of at least two target polynucleotides from multiple samples, e.g., multiple samples from multiple subjects, can be assessed sequentially or simultaneously. In some embodiments, the methylation status of at least two target polynucleotides from multiple samples, e.g., multiple samples from multiple subjects, can be assessed sequentially. For example, methylation status of at least 2, 3, 4, 5, 6, 7, 8, 9, 10, 20, 30, 40, 50, 60, 70, 80, or 83 of the isolated polynucleotides, or the isolated polynucleotides corresponding to all of Target 1 to Target 83 listed in Table 1, or a numerical range or subrange thereof, can be assessed sequentially. In other embodiments, the methylation status of at least two target polynucleotides from multiple samples, e.g., multiple samples from multiple subjects, can be assessed simultaneously. For example, methylation status of at least 2, 3, 4, 5, 6, 7, 8, 9, 10, 20, 30, 40, 50, 60, 70, 80, or 83 of the isolated polynucleotides, or the isolated polynucleotides corresponding to all of Target 1 to Target 83 listed in Table 1, or a numerical range or subrange thereof, can be assessed simultaneously.

In some embodiments, the methylation metric for each of the target polynucleotides is obtained using a computer. In other embodiments, the methylation matrix of a sample is obtained using a computer based on the methylation metrics from each of the target polynucleotides.

The present methods can be used for any suitable purpose. For example, the present methods can be used for diagnosis, prognosis, stratification, risk assessment, or treatment monitoring of colorectal cancer and/or advanced adenoma in a subject. In some embodiments, the present methods can be used for diagnosis, prognosis, stratification, risk assessment, or treatment monitoring of colorectal cancer in a subject. In other embodiments, the present methods can be used for diagnosis, prognosis, stratification, risk assessment, or treatment monitoring of advanced adenoma in a subject.

The colorectal cancer and/or advanced adenoma in multiple subjects can be assessed sequentially or simultaneously. In some embodiments, the colorectal cancer and/or advanced adenoma in multiple subjects can be assessed sequentially. For example, the colorectal cancer and/or advanced adenoma in at least 2, 3, 4, 5, 6, 7, 8, 9, 10, 20, 30, 40, 50, 60, 70, 80, 90, 100, 200, 300, 400, 500, 600, 700, 800, 900, 1,000, 1,100, 1,200, 1,300, 1,400, 1,500, 1,600, 1,700, 1,800, 1,900, 2,000, 3,000, 4,000, 5,000, 6,000, 7,000, 8,000, 9,000, 10,000 or more subjects can be assessed sequentially. In other embodiments, the colorectal cancer and/or advanced adenoma in multiple subjects can be assessed simultaneously. For example, the colorectal cancer and/or advanced adenoma in at least 2, 3, 4, 5, 6, 7, 8, 9, 10, 20, 30, 40, 50, 60, 70, 80, 90, 100, 200, 300, 400, 500, 600, 700, 800, 900, 1,000, 1,100, 1,200, 1,300, 1,400, 1,500, 1,600, 1,700, 1,800, 1,900, 2,000, 3,000, 4,000, 5,000, 6,000, 7,000, 8,000, 9,000, 10,000 or more subjects can be assessed simultaneously.

The present tests can have any suitable sensitivity. For example, the present tests can have a sensitivity of at least 10%, 20%, 30%, 40%, 50%, 60%, 70%, 80%, 90%, 91%, 92%, 93%, 94%, 95%, 96%, 97%, 98%, 99%, 99.9%, 99.999%, 99.9999%, or 100%.

The present tests can have any suitable specificity. For example, the present tests can have a specificity of at least 10%, 20%, 30%, 40%, 50%, 60%, 70%, 80%, 90%, 91%, 92%, 93%, 94%, 95%, 96%, 97%, 98%, 99%, 99.9%, 99.999%, 99.9999%, or 100%.

The present methods can further comprise treating a subject or altering treatment of a subject based on the assessment of colorectal cancer and/or advanced adenoma in the subject. For example, the present methods can further comprise treating a human patient or altering treatment of a human patient based on the assessment of colorectal cancer and/or advanced adenoma in the human patient. Exemplary treatment can be chemotherapy, radiotherapy, immunotherapy, cell therapy, surgery, treatment with a drug, e.g., a small molecule drug or a large molecule drug such an antibody drug.

Exemplary medications for treating colorectal cancer include Oxaliplatin, Avastin, Xeloda, Fluorouracil, Capecitabine, Bevacizumab, Leucovorin, Eloxatin, Methotrexate, Opdivo, Nivolumab, Yervoy, Ipilimumab, Irinotecan, Stivarga, Erbitux, Camptosar, Cetuximab, Vectibix, Lonsurf, Fusilev, Panitumumab, Zaltrap, Betaseron, Keytruda, Regorafenib, Mvasi, Braftovi, Cyramza, Pembrolizumab, Ziv-aflibercept, Interferon beta-1b, Khapzory, Levoleucovorin, Ramucirumab, tipiracil and trifluridine, Zirabev, Alymsys, and Encorafenib. In some embodiments, drugs approved for treating colon cancer include Alymsys (Bevacizumab), Avastin (Bevacizumab), Bevacizumab, Camptosar (Irinotecan Hydrochloride), Capecitabine, Cetuximab, Cyramza (Ramucirumab), Eloxatin (Oxaliplatin), Erbitux (Cetuximab), 5-FU (Fluorouracil Injection), Fluorouracil Injection, Ipilimumab, Irinotecan Hydrochloride, Keytruda (Pembrolizumab), Leucovorin Calcium, Lonsurf (Trifluridine and Tipiracil Hydrochloride), Mvasi (Bevacizumab), Nivolumab, Opdivo (Nivolumab), Oxaliplatin, Panitumumab, Pembrolizumab, Ramucirumab, Regorafenib, Stivarga (Regorafenib), Trifluridine and Tipiracil Hydrochloride, Vectibix (Panitumumab), Xeloda (Capecitabine), Yervoy (Ipilimumab), Zaltrap (Ziv-Aflibercept), Zirabev (Bevacizumab) and Ziv-Aflibercept. In some embodiments, drug combinations used in colon cancer include CAPOX, FOLFIRI, FOLFIRI-BEVACIZUMAB, FOLFIRI-CETUXIMAB, FOLFOX, FU-LV, XELIRI, and XELOX. In some embodiments, drugs approved for treating rectal cancer include Alymsys (Bevacizumab), Avastin (Bevacizumab), Bevacizumab, Camptosar (Irinotecan Hydrochloride), Capecitabine, Cetuximab, Cyramza (Ramucirumab), Eloxatin (Oxaliplatin), Erbitux (Cetuximab), 5-FU (Fluorouracil Injection), Fluorouracil Injection, Ipilimumab, Irinotecan Hydrochloride, Keytruda (Pembrolizumab), Leucovorin Calcium, Lonsurf (Trifluridine and Tipiracil Hydrochloride), Mvasi (Bevacizumab), Nivolumab, Opdivo (Nivolumab), Oxaliplatin, Panitumumab, Pembrolizumab, Ramucirumab, Regorafenib, Stivarga (Regorafenib), Trifluridine and Tipiracil Hydrochloride, Vectibix (Panitumumab), Xeloda (Capecitabine), Yervoy (Ipilimumab), Zaltrap (Ziv-Aflibercept), Zirabev (Bevacizumab), and Ziv-Aflibercept. In some embodiments, drug combinations used in rectal cancer include CAPOX, FOLFIRI, FOLFIRI-BEVACIZUMAB, FOLFIRI-CETUXIMAB, FOLFOX, FU-LV, XELIRI, and XELOX. In some embodiments, drugs approved for treating Gastroenteropancreatic Neuroendocrine Tumors include Afinitor (Everolimus), Everolimus, Lanreotide Acetate, and Somatuline Depot (Lanreotide Acetate).

The present methods can further comprise subjecting a subject to further tests, e.g., gold-standard diagnostic or prognostic test(s), based on the assessment of colorectal cancer and/or advanced adenoma in the subject. For example, the present methods can further comprise subjecting a human patient to further tests, e.g., gold-standard diagnostic or prognostic test(s), based on the assessment of colorectal cancer and/or advanced adenoma in the human patient. Exemplary test can be any suitable type of tests, e.g., in vivo test, in vitro test, tests on molecules such as DNA, RNA, protein, peptide, a complex or combination thereof, immunotest, molecular test, cellular test, tissue test, organ test or whole-body test, etc.

### E. EXEMPLARY DATA ANALYSIS METHODS

### Preprocessing

In some embodiments, sequencing reads are first adapter trimmed (to remove any synthetic adapter sequence originating from the sequencing library construction process) using a trimming program such as Cutadapt (http://journal.embnet.org/index.php/embnetjournal/article/view/200/479) or Trimmomatic (https://www.ncbi.nlm.nih.gov/pubmed/24695404). Trimmed reads are then mapped to the human reference genome using an alignment program such as Bowtie (http://bowtie-bio.sourceforge.net/index.shtml), Bowtie2 (http://bowtie-bio.sourceforge.net/bowtie2/index.shtml), or BWA (http://bio-bwa.sourceforge.net). The resultant aligned read file is then sorted and indexed using Samtools (https://github.com/samtools/samtools). Reads are then grouped into sets corresponding to each target region that can be used for methylation metric computation.

### Methylation Metric Computation

In some embodiments, for each target, a “methylation metric” representing that individual region's methylation state is computed using the aligned reads. Four metrics that can be used are Average Methylation Frequency (AMF), Methylation Haplotype Load (MHL), UnMethylation Haplotype Load (UMHL), and Percent Discordant Reads (PDR).

Average Methylation Frequency is computed by summing the number of cytosine bases observed at CpG sites within the target region and dividing by the number of cytosine and thymine bases observed at CpG sites within the target region:

$$A M F = \frac{{Σ}_{i = 1}^{N} {C}_{i}}{{Σ}_{i = 1}^{N} {C}_{i} + {T}_{i}}$$

Where N is the number of reads within the target region, i is the ith read in the target region, Cᵢ is the number of cytosines observed in the ith read, and Tᵢ is the number of thymines observed in the ith read.

Methylation Haplotype Load is computed by taking all possible substrings of adjacent CpG sites within a target region and calculating a weighted sum of the fraction of reads showing full methylation within each substring:

$$MHK = \frac{{\sum }_{{L}_{i} = 1}^{n} {L}_{i} \frac{{C}_{i}}{{N}_{i}}}{{\sum }_{{L}_{i} = 1}^{n} {L}_{i}}$$

Where n is the number of CpG sites within the target region, Lᵢ is the length of the current substring, Cᵢ is the number of reads containing all cytosines at CpG sites within substrings of length Lᵢ, and Nᵢ is the number of reads containing a substring of length Li.

Unmethylation Haplotype Load is computed by taking all possible substrings of adjacent CpG sites within a target region and calculating a weighted sum of the fraction of reads showing no methylation within each substring:

$$UMHL = \frac{{\sum }_{{L}_{i} = 1}^{n} {L}_{i} \frac{{T}_{i}}{{N}_{i}}}{{\sum }_{{L}_{i} = 1}^{n} {L}_{i}}$$

Where n is the number of CpG sites within the target region, Lᵢ is the length of the current substring, Tᵢ is the number of reads containing all thymines at CpG sites within substrings of length Lᵢ, and Nᵢ is the number of reads containing a substring of length Lᵢ.

Percent Discordant Reads is computed by taking one hundred minus the percent of reads within each region showing all cytosines or all thymines at CpG sites within the target region:

$$P D R = 100 ( 1 - \frac{{C}_{i} + {T}_{i}}{{N}_{i}} )$$

Where Nᵢ is the number of reads within the target region, Cᵢ is the number of reads containing cytosines at all covered CpG sites, and Tᵢ is the number of reads containing thymines at all covered CpG sites.

### Classification

In some embodiments, using the methylation metrics for each target region, a numerical matrix is computed (with one row for each target region, one column for each sample, and values representing the methylation metric for that region and sample). Samples are then classified using a classification algorithm such as logistic regression, k-nearest neighbors, random forest, or support vector machines.

Logistic Regression classification involves curve-fitting an equation for the probability of a sample being cancerous to the weighted sum of methylation metric values using a set of known samples. Using a least-squares regression on data from samples with known categories, the parameters for the below equation are estimated:

$$log ( \frac{p}{1 - p} ) = {β}_{0} + {\sum }_{i}^{n} {β}_{i} {M}_{i}$$

Where n is the set of target regions, i is the current target region, Mᵢ is the methylation metric value for the ith target region, βᵢ is the least-square parameter for the ith target region, and p is the probability that the sample is a cancer sample. Once the parameters for this equation are derived using known samples, an unknown sample can be classified as cancer or healthy by computing the weighted sum of methylation metric values, computing the probability using the derived equation, and comparing to a threshold.

K-nearest neighbor algorithm classifies an unknown sample using the distance of methylation metric values between the unknown sample and its k closest neighbors with known categories. First, the distances (in this case Euclidean distances) between the methylation metric values of the current sample and all other samples are computed:

$${E}_{i} = \sqrt{{\sum }_{i}^{N} {\sum }_{j}^{n} {( {M}_{j} - {m}_{i j} )}^{2}}$$

Where N is the set of known samples, i is the current known sample, n is the set of target regions, j is the current target region, Mⱼ is the methylation metric value of the unknown sample in the jth target region, mᵢⱼ is the methylation metric value of the ith known sample in the jth target region, and Eᵢ is the Euclidean distance between the current sample and the ith known sample. The categories of the k known samples with the lowest Euclidean distance are used to determine the category of the unknown sample; the category with the highest occurrence is assigned to the unknown sample.

Random Forest classification involves creating multiple decision trees using a randomly chosen subset of samples with known categories and a subset of target regions; the methylation metric values of an unknown sample are then subjected to each individual decision tree, and the majority vote is used for classification. For example, using known data, the following three decision trees might be constructed:

$${MHL}_{1} \begin{matrix}\geq 0.5 : Cancer \\ < 0.5 : Healthy\end{matrix}$$

$${MHL}_{2} \begin{matrix}\geq 0.1 : Cancer \\ < 0.1 : Healthy\end{matrix}$$

$${MHL}_{3} \begin{matrix}< 0.2 : Cancer \\ \geq 0.2 : Healthy\end{matrix}$$

An unknown sample containing the MHL values (MHL₁: 0.6, MHL₂: 0.2, MHL₃: 0.6) would then be classified as Cancer, as it would have two votes for cancer and one vote for healthy.

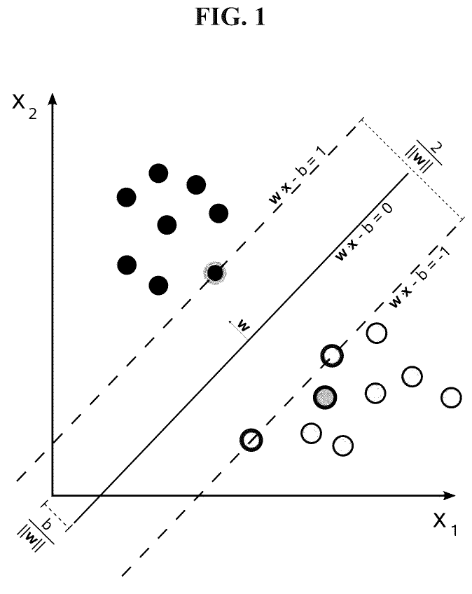

Support Vector Machines construct a hyperplane between known samples of different categories that maximizes the distance between each known sample and the hyperplane. This plane is then used as a “divider” to classify unknown samples, with samples falling on different sides of the plane being categorized differently. To illustrate an example using 2 target regions, the methylation metric values of known samples are plotted in space, and a hyperplane is drawn between the different groups (see FIG. 1), where X₁ is the methylation metric value of the first target region, X₂ is the methylation metric value of the second target region, known cancer samples are drawn in black, known healthy samples are drawn in white, and the black line represents the hyperplane. To classify an unknown sample (shown in orange), the methylation metric values are compared to the hyperplane; if the sample falls on the same side as the healthy known samples (as it does here), the sample is classified as healthy. Image of FIG. 1 is taken from (Wikipedia: https://en.wikipedia.org/wiki/Support_vector_machine) for illustration purpose only, and does not represent any DNA methylation status analysis or cancer assessment.

### F. EXEMPLARY POLYNUCLEOTIDE FRAGMENT ANALYSIS BY LIBRARY CONSTRUCTION AND POLYNUCLEOTIDE SEQUENCING

In one aspect, the target (or template) polynucleotide of the present method is a fragmented polynucleotide, for example, ranging from about 100 residues to about 1,000 residues, and in some embodiments, ranging from about 150 residues to about 400 residues.

The target or template DNA can include regular genomic DNA, chromosomal DNA, extrachromosomal DNA (such as mitochondrial DNA), or a fragment thereof. In other embodiments, the target or template DNA is a processed DNA, for example, one that has undergone enzyme digestion, cross-linking, chemical or physical shearing, bisulfite conversion, and/or degradation.

Bisulfite conversion is a method that uses bisulfite to determine the methylation pattern of DNA. DNA methylation is a biochemical process involving the addition of a methyl group to the cytosine or adenine DNA nucleotides. DNA methylation stably alters the expression of genes in cells as cells divide and differentiate from embryonic stem cells into specific tissues. In bisulfite conversion, target nucleic acids are first treated with bisulfite reagents that specifically convert un-methylated cytosines to uracils while having no impact of methylated cytosine. One consequence of bisulfite conversion is that the double-stranded conformation of the original target is disrupted due to loss of sequence complementarity. The target sequences exist as two separate single-stranded DNAs during sample preparation and analytical or diagnostic testing. Target nucleic acid sequences frequently also exist at very low concentrations. This is an especially important consideration for circulating tumor DNA (also referred to as “cell-free tumor DNA,” or “ctDNA”) due to its often low concentration in circulation and the very low variant allele fraction.

In some embodiments, the nucleic acid molecule of interest disclosed herein is a cell-free DNA, such as cell-free fetal DNA (also referred to as “cfDNA”) or ctDNA. cfDNA circulates in the body, such as in the blood, of a pregnant mother, and represents the fetal genome, while ctDNA circulates in the body, such as in the blood, of a cancer patient, and is generally pre-fragmented. In other embodiments, the nucleic acid molecule of interest disclosed herein is an ancient and/or damaged DNA, for example, due to storage under damaging conditions such as in formalin-fixed samples, or partially digested samples.

As cancer cells die, they release DNA into the bloodstream. This DNA, known as circulating tumor DNA (ctDNA), is highly fragmented, with an average length of approximately 150 base pairs. Once the white blood cells are removed, ctDNA generally comprises a very small fraction of the remaining plasma DNA, for example, ctDNA may constitute less than about 10% of the plasma DNA. Generally, this percentage is less than about 1%, for example, less than about 0.5% or less than about 0.01%. Additionally, the total amount of plasma DNA is generally very low, for example, at about 10 ng/ml of plasma.

The variants in the ctDNA can be interrogated using various methods, including next generation sequencing. Due to the low ratio of ctDNA to plasma DNA, it is difficult to call a variant with high confidence due to PCR and sequencing errors. Unique molecular identifiers (UMIs) are generally used to tag original molecules such that any variant seen can be compared to a consensus sequence. This is an effective manner to separate true from false positives. If the variant is matched to a consensus, it is a true positive. Otherwise, it is removed from analysis. Furthermore, it is essential that a high percentage of original molecules are turned into sequencing libraries so that the sensitivity remains high, i.e., variants are not missed due to dropout. Thus, ligation efficiency is extremely important during library construction.

In one aspect, provided herein is a technique to vastly improve ligation efficiency while still targeting selected regions of the genome. In one embodiment, polynucleotides to be detected by sequencing, such as ctDNA, are first dephosphorylated to remove 5′ phosphates to prevent ligation of ctDNA to itself. The ctDNA is then denatured such that all DNA is single stranded. Circligase™, a single stranded DNA ligase, is used to ligate an adapter to the 3′ end of the ctDNA. In one aspect, the adapter contains 2 specific bases on the 5′ end to optimize ligation efficiency, followed by a UMI. In one aspect, the 3′ end of the adapter contains a carbon spacer to prevent self-ligation of the adapters. In another aspect, the ligation reaction is further optimized using a crowding agent, such as PEG 4000. In one aspect, following ligation, molecules are double-stranded using a primer that is reverse complement to the adapter. This allows efficient removal of excess unligated adapters without removed usable DNA by a standard purification.

In one aspect, the DNA is then amplified using a semi-targeted PCR. One primer is reverse complement to the adapter, while the other (e.g., as one primer in a primer pool) anneals to specific, targeted regions of the genome. The specific primers were designed to minimize primer-dimer interactions and off-target annealing. In one aspect, the target-specific primers are further optimized to land in close proximity to specific variants due to the small DNA size. Following another cleanup, a PCR adds the full-length sequencing adapters and barcodes. The final library is then sequenced, for example, on an Illumina machine.

In one aspect, the semi-targeted PCR results in enrichments of >about 40,000 fold of the original molecule set despite having a relatively small target region of ~30,000 bp. In one aspect, the overall conversion rates of the present method are at least 60%, implying that at least ~3 times more of the original molecules are converted into sequenceable material when compared to standard library construction and hybridization capture. In other embodiments, the overall conversion rates are between about 60% and about 70%, between about 70% and about 80%, between about 80% and about 90%, or over 90%. In one aspect, the present method thus is able to accurately call genetic or genomic variants, such as SNVs, indels, CNVs, and fusions at extremely low mutant allele fractions, for example, as low as 0.01%. In other aspects, the allele fraction of the genetic or genomic variant is about 0.05%, about 0.1%, about 0.5%, about 1%, or about 2%.

The following sections describe certain steps of the present method in greater detail.

### Single-Stranded Polynucleotide Libraries and Method of Constructing the Same

Library construction for next generation sequencing, for example, for ctDNA, generally consists of several steps, including end repair, A-tailing, and a double stranded ligation of an adapter molecule. These ligated molecules can then be enriched 1000-2000 times at certain genomic regions using hybridization capture. Despite several improvements in library construction over the last several years, the process remains inefficient, resulting in many original molecules lost during the various steps. Double stranded ligation efficiency remains low, with ~20-30% of the molecules being properly ligated. Additionally, many molecules are lost during the purification and hybridization capture steps, so that the final conversion rate approximates 10-20%. Sensitivity remains low when interrogating low allele fraction variants found in ctDNA. This limits the accuracy when calling low allele fraction mutants, since the low efficiency will result in sensitivity loss when looking at libraries with low allele fractions.

In addition, the small size of certain polynucleotides, such as ctDNA, prevents the use of tagmentation-based library construction. For example, the polynucleotides are first tagged (e.g., with biotin) to generate a targeted library, and then enriched by capturing the tags (e.g., by streptavidin). This way, the library for the regions of interest can be enriched by about 1000-2000 fold. Finally, a PCR is performed to amplify and index the molecules for sequencing. However, PCR based methods prove difficult to add UMIs to original molecules and result in high error rates.

In one aspect, the compositions, kits, and methods described herein addressed the above problems. In some embodiments, the compositions, kits, and methods are useful in sequencing nucleic acid molecules, including but not limited to construction of various libraries, various amplification reactions (such as by PCR and/or primer extension), purification of the constructed libraries, and analysis of sequencing reads.

In certain aspects, a sequencing library can be prepared, for example, from a sample containing fragmented polynucleotides, such as fragment DNA. In one aspect, the sample is obtained a naturally occurring sample, for example, directly from a subject, such as tissue fluid or body fluid, including but not limited to blood, plasma, serum, cerebrospinal fluid, synovial fluid, urine, sweat, semen, sputum, tear, mucus, or amniotic fluid. In other aspects, a sequencing library can be prepared by forming fragments of DNA (for example, by shearing the DNA), and attaching the adapters herein to the DNA fragments. In particular embodiments, the fragmented polynucleotides and the adapters are single-stranded.

The fragments (for example, the ctDNA or fragments formed by fragmenting longer DNA strands) are sometimes referred to as “inserts,” as they can be “inserted” or ligated adjacent to an adapter such as a single-stranded adaptor disclosed herein. RNA molecules can also be sequenced, for example by reverse transcribing the RNA molecules to form DNA molecules, which are attached to the adapters.

In one aspect, a method comprising ligating a set of adaptors to a library of single-stranded polynucleotides is provided, and in the method, the ligation is catalyzed by a single-stranded DNA (ssDNA) ligase. As used herein, a ssDNA ligase is capable of ligating ends of ssDNA in the absence of a complementary sequence. For example, CircLigase™ ssDNA Ligase and CircLigase™ II ssDNA Ligase are both thermostable ligases that are typically used to catalyze intramolecular ligation (i.e., circularization) of ssDNA templates having a 5′-phosphate and a 3′-hydroxyl group. In contrast to T4 DNA Ligase and Ampligase® DNA Ligase, which ligate DNA ends that are annealed adjacent to each other on a complementary DNA sequence, a ssDNA ligase ligates ends of ssDNA in the absence of a complementary sequence. The enzyme is therefore useful for making circular ssDNA molecules from linear ssDNA. Circular ssDNA molecules can be used as substrates for rolling-circle replication or rolling-circle transcription. In addition to its activity on ssDNA, a CircLigase enzyme also has activity in ligating a single-stranded nucleic acid having a 3′-hydroxyl ribonucleotide and a 5′-phosphorylated ribonucleotide or deoxyribonucleotide.

Either CircLigase™ ssDNA Ligase or CircLigase™ II ssDNA Ligase can be used in the present disclosure. The two enzymes are different in that CircLigase I is far less adenylated than CircLigase II and requires ATP for best activity. CircLigase I recircularizes ssDNA in the presence of ATP. CircLigase II is nearly 100% adenylated, therefore it is not necessary to add ATP to the reaction buffer. CircLigase II works as a stoichiometric reaction, where the enzyme bonds the 5′-end of an oligo that is adenylated in the enzyme active site, and then ligates the oligo and stops. Since the reaction doesn't contain ATP, CircLigase II works in a 1:1 enzyme: oligo configuration. Once the circularization is complete, the circular ssDNA is released from the active site and the reaction stops.

In one aspect, each single-stranded polynucleotide is blocked at the 5′ end to prevent ligation at the 5′ end, each adaptor comprises a unique molecular identifier (UMI) sequence that earmarks the single-stranded polynucleotide to which the adaptor is ligated, each adaptor is blocked at the 3′ end to prevent ligation at the 3′ end, and the 5′ end of the adaptor is ligated to the 3′ end of the single-stranded polynucleotide by the ssDNA ligase to form a linear ligation product, thereby obtaining a library of linear, single-stranded ligation products. Template-independent circularization of single-stranded DNA is described in WO2010/094040 A1, the disclosure of which is incorporated herein in its entirety. WO2010/094040 A1, however, only discloses intramolecular ligation (e.g., circularization) of single-stranded polynucleotides.

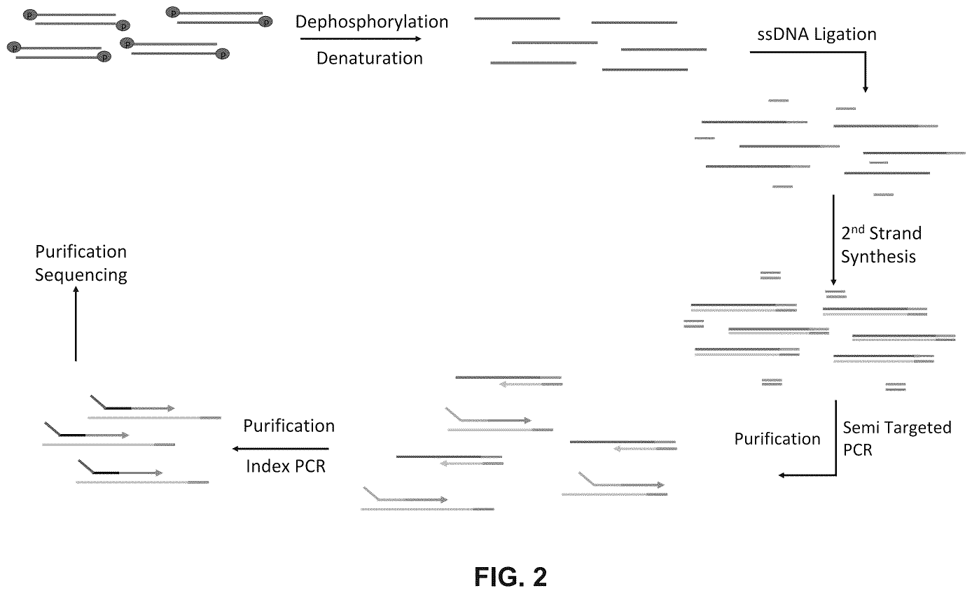

Thus, the present method uses a ssDNA ligase, such as CircLigase or CircLigase II, in an unconventional manner. Instead of circularization, the present ligation method aims to generate a linear ligation product between the single-stranded target polynucleotide and an adaptor molecule. In one aspect, the present disclosure uses a ssDNA ligase to carry out intramolecular ligate, e.g., for ligating an adaptor to single-stranded polynucleotides. In order to do, in one aspect, the single-stranded polynucleotide is blocked at the 5′ end to prevent circularization. This way, intramolecular ligation of the 3′ end of an ssDNA to its own 5′ end, as well as intermolecular ligation of the 3′ end of one ssDNA to the 5′ end of another ssDNA within the same library, is prevented. Thus, in one aspect, both circularization of the single-stranded polynucleotide and formation of linear concatemers (containing the single-stranded polynucleotides and/or the adaptors) are prevented during the ligation reaction. As shown in FIG. 2, the blocking of each single-stranded polynucleotide can comprise dephosphorylation at its 5′ end to prevent ligation at that end.

In another aspect, each adaptor is blocked at the 3′ end to prevent ligation at the 3′ end. This way, intramolecular ligation of the 3′ end of an adaptor to its own 5′ end, as well as intermolecular ligation of the 3′ end of one adaptor molecule to the 5′ end of another adaptor molecule, is prevented. The blocking of each adaptor can comprise a carbon spacer, ddCTP, ddATP, ddTTP, ddGTP, hexanediol, triethylene glycol (TEG), and/or hexaethylene glycol, to prevent ligation at its 3′ end. Thus, in one aspect, both circularization of the single-stranded adaptor and formation of linear concatemers (containing the single-stranded polynucleotides and/or the adaptors) are prevented during the ligation reaction.

The adaptor may comprise one or more copies of one or more spacers, in any suitable combination. For example, Gansauge and Meyer disclosed an adaptor that comprises ten copies of a C3Spacer and a biotinylated TEG spacer. Gansauge and Meyer (2013), “Single-stranded DNA library preparation for the sequencing of ancient or damaged DNA,” Nature Protocols, 8 (4): 737-48, which is incorporated herein by reference in its entirety. This reference, however, requires capturing the ligated ssDNA, via biotin-streptavidin interaction, immediately after ligation. This step may cause a significant loss of the ssDNA molecules in the library. The reference then converts the captured ssDNA to dsDNA while the ssDNA remains captured on a bead.

As shown in FIG. 2, the present disclosure does not require capturing the ligated ssDNA immediately after ligation. Instead, the ligated ssDNA remains in the ligation reaction volume when it is converted into dsDNA.

In one aspect, the ligation efficiency of the ssDNA in the library is high, for example, at least about 40%, at least about 50%, at least about 60%, at least about 70%, at least about 80%, at least about 85%, at least about 90%, at least about 95%, or at least about 99% of the single-stranded polynucleotides in the sample are ligated to an adaptor. In particular embodiments, the ligation efficiency is about 80%. With this vastly improved ligation efficiency, the presently claimed method is still capable of targeting selected regions of the genome, as explained below.

In one aspect, the adaptor has the following structure: /5′Phos/NIN2 . . . Nᵢ-UMI-M₁M₂ . . . Mⱼ-Blocker, wherein “5′Phos” represents a 5′ phosphate group, “NIN2 . . . . Nᵢ” represents the sequence 5′ to the UMI sequence, “M₁M₂ . . . Mⱼ” represents the sequence 3′ to the UMI sequence, and “Blocker” indicates that the 3′ end of the adaptor is blocked to prevent ligation thereto. Both i and j are integers, wherein i can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or greater than 30; and j can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or greater than 50. In specific embodiments, i can be 2. In some embodiments, the dinucleotide sequence N₁N₂ at the 5′ end of N₁N₂ . . . . Nᵢ can be GA (5′ to 3′), GG (5′ to 3′), AA (5′ to 3′), or AG (5′ to 3′), in order to enhance the ligation efficiency.

In one aspect, a portion or all of the M₁M₂, . . . Mⱼ sequence is used in later steps for designing a reverse-complement sequence that is used as a primer to convert the ligated single-stranded polynucleotide into a double-stranded polynucleotide, and/or for the semi-targeted PCR to amplify a selected target sequence (the other primer of the primer pair being the target-specific primer). In one aspect, the M₁M₂ . . . Mⱼ sequence comprises AGATCGGAAGAGCGTCGTGTAGGGAAAGAGTG (SEQ ID NO:85) or a portion thereof that comprises between about 18 and 22 nucleotide residues.

In another aspect, the “Blocker” comprises a carbon spacer, ddCTP, ddATP, ddTTP, ddGTP, hexanediol, triethylene glycol (TEG), and/or hexaethylene glycol, in one or more copies of one or more blocker groups in any suitable combination and order in the 5′ to 3′ direction.

In one aspect, use of the UMI facilitates the determination, selection, and/or isolation of error-free sequencing reads of a target sequence, and the sequencing reads can be selected with high accuracy and high throughput. Such validated, error-free sequencing reads are useful in any technique that requires sequence fidelity, including the construction of larger molecules of known sequence, polymorphism and/or mutation screening, massively parallel sequencing, and quantification methods to preclude bias in the methodologies.

In one aspect, the Unique Molecular Identifier is associated with and uniquely identifies a ligated construct comprising a single-stranded target polynucleotide and an adaptor. In other words, two single-stranded target polynucleotides having the same sequence may be ligated to two different adaptors which differ from each other at their UMI sequences; the resultant ligation products are different, and each ligation product (rather than the target polynucleotides having the same sequence) is uniquely identified by the UMI. In another aspect, when the single-stranded ligation products are converted into double-stranded polynucleotides and amplified, amplification errors may be introduced during repeated copying even though very high fidelity polymerases are available. As a result, even a low error rate can have a significant impact, particularly in the construction of large libraries. Although massively parallel sequencing has advantages in cost and throughput, the accuracy of the reads can be comprised by the limitations of the amplification and/or detection technologies.

By using the UMI, the present method is capable of identifying error-free amplification products and/or sequencing reads, and excluding those with technical errors from analysis. The amplification products and/or sequencing reads having the same UMI can be confirmed as related (identical by descent), and thus sequence differences between molecules with the same UMI can be identified as technical errors rather than real differences in the sequence (e.g., sequence differences between a wild-type sequence and a cancer-related mutant sequence). In other words, since each single-stranded ligation product is unique identifiable by its UMI, all of its descendants (due to amplification and/or sequencing) should have the same target sequence if no technical error is introduced. If, however, an error such as a single-nucleotide insertion is introduced into the target sequence during amplification and/or sequencing, some amplification products and/or sequencing reads identical by descent (e.g., sharing the same UMI) will have the insertion while the others will not. The exact ratio between the products having the insertion and those that do not have the insert will vary, depending on when the error occurs during the amplification and/or sequencing process. In general, when very high fidelity polymerases are used, the products without errors will be in the majority. In another aspect, because amplification products and/or sequencing reads that are identical by descent can be identified, a consensus sequence can be determined using data from multiple molecules, thereby achieving a high accuracy for high throughput sequencing.

In one aspect, the UMI is a degenerate nucleic acid sequence, and the number of nucleotides in the UMI is designed such that the number of potential and actual sequences represented by the UMI sequences is greater than the total number of target single-stranded target polynucleotide in the initial library. In one aspect, UMI sequence diversity (or “uniqueness” with regard to each single UMI sequence) can be provided by using a degenerate collection of sequences randomly generated by synthesizing with a mixture of all four bases at each position. Alternatively, a diverse but pre-defined set of sequences can be synthesized and ligated to the initial single-stranded polynucleotide library. The diversity of the UMI set needs to be sufficient so that molecules that are not related by descent won't be mistaken as such. In one aspect, a “unique” molecular identifier need not be absolutely unique, and may be used on different target single-stranded polynucleotides provided it is clear that they are different and not mistaken for a molecule that is identical by descent. The large number of UMI sequences that can be generated from the random assembly of nucleotides provides a high probability that each individual ligation product can be uniquely identified. For example, if the UMI comprises a 12-mer synthesized with a mixture of A, C, G and T at each position, there are 4¹² possible sequences. If the UMI comprises a 20-mer synthesized with a mixture of A, C, G and T at each position, there are 4²⁰ (about 10¹²) possible sequences. The use of such random identifiers allows a large library with single-stranded target polynucleotides that can be individually distinguished from each other.

In particular aspects, the UMI is a 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer, or even longer degenerate sequence. In one aspect, the adaptor has the following structure:/5′Phos/GANNNNNNNNNNNNAGATCGGAAGAGCGTCGTGTAGGGAAAGAGTG/3SpC3/, wherein “NNNNNNNNNNNN” represents a 12-mer UMI sequence, and “3SpC3” represents a 3′ carbon spacer. The sequence of

GANNNNNNNNNNNNAGATCGGAAGAGCGTCGTGTAGGGAAAGAGTG is
SEQ ID NO: 86.

The concentration of DNA can be artificially increased by adding condensing agents such as cobalt hexamine and biogenic polyamines such as spermidine, or by using crowding agents such as polyethylene glycol (PEG) which also increase the effective concentration of enzymes. In one aspect, additives such as cobalt hexamine can produce exclusively intermolecular reaction, resulting in linear ligation products rather than circular products. Thus, in case the 5′ ends of the single-stranded target polynucleotides and the 3′ ends of the single-stranded adaptor may not be completely blocked to prevent ligation, additives such as cobalt hexamine may be used to enhance intermolecular reaction and further prevent circularization of the single-stranded target polynucleotide and/or the adaptor.

In some embodiments, more than one configurations of the adaptor can be used in the same ligation reaction. For example, two configurations of the adaptor may be used:
Configuration No. 1: /5′Phos/N₁N₂ . . . Nᵢ-UMI₁-M₁M₂ . . . . Mⱼ-Blocker₁, and
Configuration No. 2: /5′Phos/P₁P₂ . . . Pₖ-UMI₂-Q₁Q₂ . . . . Qₗ-Blocker₂.

N₁N₂ . . . Nᵢ and P₁P₂ . . . Pₖ can be the same or different, UMI₁ and UMI₂ can be the same or different, M₁M₂ . . . . Mⱼ and Q₁Q₂ . . . . Qₗ, can be the same or different, and Blocker₁ and Blocker₂ can be the same or different. In one embodiment, UMI₁ is different from UMI₂ (for example, UMI₁ is a 12-mer degenerate sequence while UMI₂ is a 13-mer degenerate sequence), while the other features of the adaptors are the same. In another embodiment, N₁N₂ . . . Nᵢ is different from P₁P₂ . . . . Pₖ (for example, one is AG while the other is GA), while the other features of the adaptors are the same. In yet another embodiment, M₁M₂ . . . . Mⱼ is different from Q₁Q₂ . . . . Qₗ, while the other features of the adaptors are the same. In still another embodiment, Blocker₁ and Blocker₂ are different, while the other features of the adaptors are the same.

After the ligation reaction, the single-stranded ligation products, without any need for purification (e.g., separation of the ligation products from the excess, unligated adaptor molecules), can be immediately subject to conversion into double-stranded ligation products. In addition, neither the single-stranded target polynucleotide nor the adaptor needs to be captured on a solid support (e.g., by biotin-streptavidin mediated binding to a bead) in order for the subsequent conversion of the ligation product into a double-stranded polynucleotide and/or amplification step. Thus, the present method avoids and/or reduces loss of the already small allele fraction of the mutant in a DNA sample, such as ctDNA, due to the purification or isolation of the single-stranded ligation products. Instead, in one aspect, the single-stranded ligation products remain in the solution which is directed subject to primer extension.

### Conversion of Single-Stranded Polynucleotide Library to Double-Stranded Polynucleotide Library

In one aspect as shown in FIG. 2, following construction of the library containing the single-stranded ligation products, the method can further comprise converting the library of linear, single-stranded ligation products into a library of linear, double-stranded ligation products. In one aspect, the conversion uses a primer or a set of primers each comprising a sequence that is reverse-complement to the adaptor and/or hybridizable to the adaptor.

For an adaptor having the following structure: /5′Phos/N₁N₂ . . . Nᵢ-UMI-M₁M₂ . . . Mⱼ-Blocker, the primer can comprise a sequence that is reverse-complement and/or hybridizable to M₁M₂ . . . Mⱼ. In this example, when the primer hybridizes to the ligated product having the structure ssDNA-N₁N₂ . . . Nᵢ-UMI-M₁M₂ . . . Mⱼ-Blocker, the primer extension reaction can convert the ssDNA-N₁N₂ . . . Nᵢ-UMI sequence (and optionally, all or a portion of the M₁M₂ . . . . Mⱼ sequence) into double-stranded polynucleotides. In one specific example, a reverse-complement primer comprises the sequence set forth in SEQ ID NO:84, CACTCTTTCCCTACACGACGC (5′ to 3′).

In some embodiments, the primer may not be a perfect reverse-complement of M₁M₂ . . . Mⱼ or a portion therefore; nonetheless, the primer is hybridizable to M₁M₂ . . . . Mⱼ (and thus the ssDNA ligated to the adaptor) under stringent conditions.

In any of the preceding embodiments, the method can further comprise amplifying and/or purifying the library of linear, double-stranded ligation products. In one aspect, the double-stranded ligation products are purified and size selected to remove unbound adaptor molecules and/or unbound primers, and/or complexes formed between an adaptor and its reverse-complement primer. Any suitable methods can be used to remove these fragments which are generally shorter than the desired double-stranded ligation products. For example, using PCR purification column from Qiagen could help to eliminate the smaller fragments from the samples and running the column-purified samples on 2% certified low range ultra agarose gel can help to select the desired fragment size. The beads-based DNA purification including AMPure method is also helpful to remove the smaller fragments. In some embodiments, the desired double-stranded ligation products size is from about 100 bps to about 600 bps, such as from about 100 bps to about 400 bps, from about 150 bps to about 200 bps, from about 200 bps to about 250 bps, and from about 250 bps to about 300 bps. In one embodiment, dsDNA (>150 bps and <400 bps) is purified and collected, for example, by eluting beads suspended in a Tri-EDTA buffer.

In one aspect, the purification is bead-based. In another aspect, the purification is based on size selection, for example, the purification step selectively purifies polynucleotides between about 50 nucleotides and about 1000 nucleotides in lengths, for example, adaptors of about 40 nucleotides in length (and primer dimers and/or primer-adaptor duplexes of about 40 bp) are removed. In one aspect, the purification is column-based, for example, by using a dsDNA or ssDNA purification column, such as those from Zymo or Qiagen.

In another aspect, the purification does not comprise using a specific binding pair (such as biotin/streptavidin), one of which is attached to the linear, double-stranded ligation product and the other is attached to a solid support (such as a bead).

In any of the preceding embodiments, the method herein can further comprise amplifying the library of linear, double-stranded ligation products, e.g., by a polymerase chain reaction (PCR), to obtain an amplified library of linear, double-stranded ligation products comprising sequence information of a target sequence. This amplification can be an unbiased amplification, for example, by ligating a universal adaptor pair to the ends of the double-stranded ligation products, and amplifying all the tagged double-stranded ligation products with a universal primer pair. In other embodiments, a semi-targeted amplification is conducted in lieu of or in addition to the unbiased amplification. The semi-targeted amplification can be performed before or after the unbiased amplification.

### Semi-Targeted Amplification of Double-Stranded Polynucleotide Library

In one aspect, as shown in FIG. 2, a semi-targeted amplification of the double-stranded ligation product library comprises using a primer comprising a sequence that is reverse-complement and/or hybridizable to the adaptor, and a primer hybridizable to a target sequence (e.g., a Target listed in Table 1) or primers hybridizable to the same target sequence or multiple target sequences.

For an adaptor having the following structure: /5′Phos/N₁N₂ . . . Nᵢ-UMI-M₁M₂ . . . Mⱼ-Blocker, the primer can comprise a sequence that is reverse-complement and/or hybridizable to M₁M₂ . . . . Mⱼ. This way, when the primer hybridizes to one strand of the dsDNA and the target-specific primer hybridizes to the other strand of the dsDNA, the PCR product will contain a target sequence as well as the N₁N₂ . . . Nᵢ-UMI sequence (and optionally, all or a portion of the M₁M₂ . . . Mⱼ sequence). In one specific example, a reverse-complement primer comprises the sequence set forth in SEQ ID NO:84, CACTCTTTCCCTACACGACGC (5′ to 3′).

In one aspect, a plurality of target-specific primers can be used, each comprising a sequence specific for the same or a different target sequence. In other words, the primers can have the same or different target sequences. In some embodiments, the pool of target-specific primers comprises about 5, about 10, about 25, about 50, about 100, about 150, about 200, about 250, about 300, about 400, about 500, about 600, about 700, about 800, about 900, about 1000, or more than about 1000 different primers, such as about 104, about 105, about 106, or more primers. In other embodiments, the pool comprises between about 20 and about 60, between about 60 and about 100, between about 100 and about 140, between about 140 and about 180, between about 180 and about 220, between about 220 and about 260, between about 260 and about 300, between about 300 and about 350, or between about 350 and about 400 different primers. In one aspect, the pool of target-specific primers are used together with one common reverse-complement primer, wherein the common reverse-complement primer forms a primer pair with each individual target-specific primer in the pool to amplify the target sequence in and/or between the primers in a semi-targeted fashion. Thus, in this aspect, the semi-targeted amplification is not a whole genome amplification.

Since ctDNA fragments randomly, in one aspect, the primer position of the target-specific primer may be important. For example, if the primer landing spans a break point, it may result in lower conversion rates. A larger target-specific primer pool and/or using multiple partially overlapping primers for the same target sequence may solve the problem.

In one aspect, the sequence information of the target sequence can comprise a mutation, a single nucleotide polymorphism (SNP), a copy number variation (CNV), or an epigenetic change. In one aspect, the mutation comprises a point mutation, an insertion, a deletion, an inversion, a truncation, a fusion, an amplification, or any combination thereof.

In some embodiments, the amplified library of linear, double-stranded ligation products can be a library other than a whole genome library, for example, a semi-targeted genome library.

In some embodiments, the method can further comprise purifying the amplified library of linear, double-stranded ligation products. Any suitable methods can be used to remove smaller fragments including primer dimers. For example, using PCR purification column from Qiagen could help to eliminate the smaller fragments from the samples and running the column-purified samples on 2% certified low range ultra agarose gel can help to select the desired fragment size. The beads-based DNA purification including AMPure method is also helpful to remove the smaller fragments. In some embodiments, the amplification product size is from about 100 bps to about 600 bps, such as from about 100 bps to about 400 bps, from about 150 bps to about 200 bps, from about 200 bps to about 250 bps, and from about 250 bps to about 300 bps. In one embodiment, dsDNA (>150 bps and <400 bps) is purified and collected, for example, by eluting beads suspended in a Tri-EDTA buffer.

In one aspect, the purification is bead-based. In another aspect, the purification is based on size selection, for example, the purification step selectively purifies polynucleotides greater about 150 nucleotides in lengths. In another aspect, the purification does not comprise using a specific binding pair (such as biotin/streptavidin), one of which is attached to the linear, double-stranded ligation product and the other is attached to a solid support (such as a bead). In one aspect, the purification is column-based, for example, by using a dsDNA or ssDNA purification column, such as those from Zymo or Qiagen.

### Construction of Sequence Library and Analysis of Sequencing Reads

In one aspect, the method further comprises sequencing the purified amplified library of linear, double-stranded ligation products. In one aspect, the sequencing step comprises attaching a sequencing adapter and/or a sample-specific barcode to each linear, double-stranded ligation product. In one particular aspect, the attaching step is performed using a polymerase chain reaction (PCR).

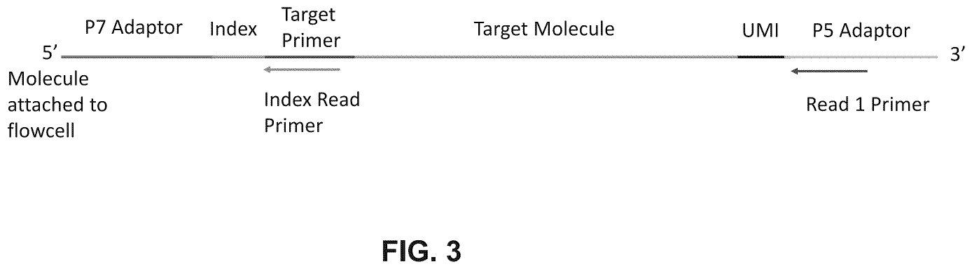

FIG. 3 shows an exemplary configuration of a construct comprising a target molecule for sequencing. For Illumina sequencing, on each end, these constructs have flow cell binding sites, P5 and P7, which allow the library fragment to attach to the flow cell surface. The P5 and P7 regions of single-stranded library fragments anneal to their complementary oligos on the flowcell surface. The flow cell oligos act as primers and a strand complementary to the library fragment is synthesized. Then, the original strand is washed away, leaving behind fragment copies that are covalently bonded to the flowcell surface in a mixture of orientations. Copies of each fragment are then generated by bridge amplification, creating clusters. Then, the P5 region is cleaved, resulting in clusters containing only fragments which are attached by the P7 region. This ensures that all copies are sequenced in the same direction. The sequencing primer anneals to the P5 end of the fragment, and begins the sequencing by synthesis process. Index reads are performed when a sample is barcoded. When Read 1 is finished, everything from Read 1 is removed and an index primer is added, which anneals at the P7 end of the fragment and sequences the barcode. Then, everything is stripped from the template, which forms clusters by bridge amplification as in Read 1. This leaves behind fragment copies that are covalently bonded to the flowcell surface in a mixture of orientations. This time, P7 is cut instead of P5, resulting in clusters containing only fragments which are attached by the P5 region. This ensures that all copies are sequenced in the same direction (opposite Read 1). The sequencing primer anneals to the P7 region and sequences the other end of the template.

Next-generation sequencing platforms, such as MiSeq (Illumina Inc., San Diego, CA), can be used for highly multiplexed assay readout. A variety of statistical tools, such as the Proportion test, multiple comparison corrections based on False Discovery Rates (see Benjamini and Hochberg, 1995, Journal of the Royal Statistical Society Series B (Methodological) 57, 289-300), and Bonferroni corrections for multiple testing, can be used to analyze assay results. In addition, approaches developed for the analysis of differential expression from RNA-Seq data can be used to reduce variance for each target sequence and increase overall power in the analysis. See Smyth, 2004, Stat. Appl. Genet. Mol. Biol. 3, Article 3.

Overall, in some embodiments, the conversion rate of the present method is at least about 40%, at least about 50%, at least about 60%, at least about 70%, at least about 80%, at least about 90%, or at least about 95%. In one aspect, the conversion rate is the percentage of targeted single-stranded polynucleotides in the initial library that give rise to sequencing reads.

In any of the preceding embodiments, the method can be used for the diagnosis and/or prognosis of a disease or condition in a subject, predicting the responsiveness of a subject to a treatment, identifying a pharmacogenetics marker for the disease/condition or treatment, and/or screening a population for a genetic information. In one aspect, the disease or condition is a cancer or neoplasia, and the treatment is a cancer or neoplasia treatment.

Mutant DNA molecules offer unique advantages over cancer-associated biomarkers because they are so specific. Though mutations occur in individual normal cells at a low rate (about 10⁻⁹ to 10⁻¹⁰ mutations/bp/generation), such mutations represent such a tiny fraction of the total normal DNA that they are orders of magnitude below the detection limit of certain art methods. Several studies have shown that mutant DNA can be detected in stool, urine, and blood of CRC patients (Osborn and Ahlquist, Stool screening for colorectal cancer: molecular approaches, Gastroenterology 2005; 128:192-206).

Based on the sequencing results herein, detection of circulating tumor DNA in the patient can be made, and diagnosis of cancer and predictions regarding tumor recurrence can be made. Based on the predictions, treatment and surveillance decisions can be made. For example, circulating tumor DNA which indicates a future recurrence, can lead to additional or more aggressive therapies as well as additional or more sophisticated imaging and monitoring. Circulating DNA refers to DNA that is ectopic to a tumor.

Samples which can be monitored for ctDNA include blood and stool. Blood samples may be for example a fraction of blood, such as serum or plasma. Similarly stool can be fractionated to purify DNA from other components. Tumor samples are used to identify a somatically mutated gene in the tumor that can be used as a marker of tumor in other locations in the body. Thus, as an example, a particular somatic mutation in a tumor can be identified by any standard means known in the art. Typical means include direct sequencing of tumor DNA, using allele-specific probes, allele-specific amplification, primer extension, etc. Once the somatic mutation is identified, it can be used in other compartments of the body to distinguish tumor derived DNA from DNA derived from other cells of the body. Somatic mutations are confirmed by determining that they do not occur in normal tissues of the body of the same patient. Types of tumors which can be diagnosed and/or monitored in this fashion are virtually unlimited. Any tumor which sheds cells and/or DNA into the blood or stool or other bodily fluid can be used. Such tumors include, in addition to colorectal tumors, tumors of the breast, lung, kidney, liver, pancreas, stomach, brain, head and neck, lymphatics, ovaries, uterus, bone, blood, etc.

In one aspect, the method disclosed herein can be used to construct a library for use in sequencing and/or in determining an epigenetic status/state of one or more regions of the target sequence. DNA methylation was first the discovered epigenetic mark. Epigenetics is the study of changes in gene expression or cellular phenotype caused by mechanisms other than changes in the underlying DNA sequence. Methylation predominately involves the addition of a methyl group to the carbon-5 position of cytosine residues of the dinucleotide CpG and is associated with repression or inhibition of transcriptional activity.

Bisulfite conversion is the use of bisulfite reagents to treat DNA to determine its pattern of methylation. The treatment of DNA with bisulfite converts cytosine residues to uracil but leaves 5-methylcytosine residues unaffected. Thus, bisulfite treatment introduces specific changes in the DNA sequence that depend on the methylation status of the individual cytosine residues. Various analyses can be performed on the altered sequence to retrieve this information, for example, in order to differentiate between single nucleotide polymorphisms (SNP) resulting from the bisulfite conversion. U.S. Pat. Nos. 7,620,386, 9,365,902, and U.S. Patent Application Publication 2006/0134643, all of which are incorporated herein by reference, exemplify methods known to one of ordinary skill in the art with regard to detecting sequences altered due to bisulfite conversion.

As discussed above, one consequence of bisulfite conversion is that the double-stranded conformation of the original target is disrupted due to loss of sequence complementarity. While this may cause problem for traditional methods for constructing double-stranded libraries, in one aspect the present method is uniquely suited to construct single-stranded libraries from bisulfite conversion sample for sequencing analysis.

In another aspect, the present method can be used in combination with a method for determining a methylation state/status, for example, as described in U.S. Provisional Application No. 62/487,422, entitled “Compositions and Methods for Detection of Genomic Variance and DNA Methylation Status,” filed Apr. 19, 2017, and US 2020/0048697 A1, which are incorporated herein by reference in their entireties for all purposes. In one embodiment, a sample is contacted with a methylation-sensitive restriction enzyme (MSRE) before the dephosphorylation and/or the denaturing step, and methylation profiles are then be analyzed by constructing a single-stranded library by ligation as disclosed herein.

### Further Exemplary Embodiments

In any of the preceding embodiments, the ssDNA ligase can be a Thermus bacteriophage RNA ligase such as a bacteriophage TS2126 RNA ligase (e.g., CircLigase™ and CircLigase II™), or an archaebacterium RNA ligase such as Methanobacterium thermoautotrophicum RNA ligase 1. In other aspects, the ssDNA ligase is an RNA ligase, such as a T4 RNA ligase, e.g., T4 RNA ligase I, e.g., New England Biosciences, M0204S, T4 RNA ligase 2, e.g., New England Biosciences, M0239S, T4 RNA ligase 2 truncated, e.g., New England Biosciences, M0242S, T4 RNA ligase 2 truncated KQ, e.g., M0373S, or T4 RNA ligase 2 truncated K227Q, e.g., New England Biosciences, M0351S. In any of the preceding embodiments, the kit can also comprise a thermostable 5′ App DNA/RNA ligase, e.g., New England Biosciences, M0319S, or T4 DNA ligase, e.g., New England Biosciences, M0202S.

In some embodiments, the present methods comprise ligating a set of adaptors to a library of single-stranded polynucleotides using a single-stranded DNA (ssDNA) ligase. Any suitable ssDNA ligase, including the ones disclosed herein, can be used. The adaptors can be used at any suitable level or concentration, e.g., from about 1 μM to about 100 μM such as about 1 μM, 10 μM, 20 μM, 30 μM, 40 μM, 50 μM, 60 μM, 70 μM, 80 μM, 90 μM, or 100μ, or any subrange thereof. The adapter can comprise or begin with any suitable sequences or bases. For example, the adapter sequence can begin with all 2 bp combinations of bases.

In some embodiments, the ligation reaction can be conducted in the presence of a crowding agent. In one aspect, the crowding agent comprises a polyethylene glycol (PEG), such as PEG 4000, PEG 6000, or PEG 8000, Dextran, and/or Ficoll. The crowding agent, e.g., PEG, can be used at any suitable level or concentration. For example, the crowding agent, e.g., PEG, can be used at a level or concentration from about 0% (w/v) to about 25% (w/v), e.g., at about 0% (w/v), 1% (w/v), 2% (w/v), 3% (w/v), 4% (w/v), 5% (w/v), 6% (w/v), 7% (w/v), 8% (w/v), 9% (w/v), 10% (w/v), 11% (w/v), 12% (w/v), 13% (w/v), 14% (w/v), 15% (w/v), 16% (w/v), 17% (w/v), 18% (w/v), 19% (w/v), 20% (w/v), 21% (w/v), 22% (w/v), 23% (w/v), 24% (w/v), or 25% (w/v), or any subrange thereof.

In some embodiments, the ligation reaction can be conducted for any suitable length of time. For example, the ligation reaction can be conducted for a time from about 2 to about 16 hours, %, e.g., for about 2 hours, 3 hours, 4 hours, 5 hours, 6 hours, 7 hours, 8 hours, 9 hours, 10 hours, 11 hours, 12 hours, 13 hours, 14 hours, 15 hours, or 16 hours, or any subrange thereof.

In some embodiments, the ssDNA ligase in the ligation reaction can be used in any suitable volume. For example, the ssDNA ligase in the ligation reaction can be used at a volume from about 0.5 μl to about 2 μl, %, e.g., at about 0.5 μl, 0.6 μl, 0.7 μl, 0.8 μl, 0.9 μl 1 μl, 1.1 μl, 1.2 μl, 1.3 μl, 1.4 μl, 1.5 μl, 1.6 μl, 1.7 μl, 1.8 μl, 1.9 μl, or 2 μl, or any subrange thereof.

In some embodiments, the ligation reaction can be conducted in the presence of a ligation enhancer, e.g., betaine. The ligation enhancer, e.g., betaine, can be used at any suitable volume, e.g., from about 0 μl to about 1 μl, e.g., at about 0 μl, 0.1 μl, 0.2 μl, 0.3 μl, 0.4 μl, 0.5 μl, 0.6 μl, 0.7 μl, 0.8 μl, 0.9 μl, 1 μl, or any subrange thereof.

In some embodiments, the ligation reaction can be conducted using a T4 RNA ligase I, e.g., the T4 RNA ligase I from New England Biosciences, M0204S, in the following exemplary reaction mix (20 μl): 1× Reaction Buffer (50 mM Tris-HCl, pH 7.5, 10 mM MgCl2, 1 mM DTT), 25% (wt/vol) PEG 8000, 1 mM hexamine cobalt chloride (optional), 1 μl (10 units) T4 RNA Ligase, and 1 mM ATP. The reaction can be incubated at 25° C. for 16 hours. The reaction can be stopped by adding 40 μl of 10 mM Tris-HCl pH 8.0, 2.5 mM EDTA.

In some embodiments, the ligation reaction can be conducted using a Thermostable 5′ App DNA/RNA ligase, e.g., the Thermostable 5′ App DNA/RNA ligase from New England Biosciences, M0319S, in the following exemplary reaction mix (20 μl): ssDNA/RNA Substrate 20 μmol (1 μmol/ul), 5′ App DNA Oligonucleotide 40 μmol (2 μmol/ul), 10× NEBuffer 1 (2 ul), 50 mM MnCl2 (for ssDNA ligation only) (2 μl), Thermostable 5′ App DNA/RNA Ligase (2 ul (40 μmol)), and Nuclease-free Water (to 20 μl). The reaction can be incubated at 65° C. for 1 hour. The reaction can be stopped by heating at 90° C. for 3 minutes.

In some embodiments, the ligation reaction can be conducted using a T4 RNA ligase 2, e.g., the T4 RNA ligase 2 from New England Biosciences, M0239S, in the following exemplary reaction mix (20 μl): T4 RNA ligase buffer (2 μl), enzyme (1 μl), PEG (10 μl), DNA (1 μl), Adapter (2 μl), and water (4 μl). The reaction can be incubated at 25° C. for 16 hours. The reaction can be stopped by heating at 65° C. for 20 minutes.

In some embodiments, the ligation reaction can be conducted using a T4 RNA ligase 2 Truncated, e.g., the T4 RNA ligase 2 Truncated from New England Biosciences, M0242S, in the following exemplary reaction mix (20 μl): T4 RNA ligase buffer (2 μl), enzyme (1 μl), PEG (10 μl), DNA (1 μl), Adapter (2 μl), and water (4 μl). The reaction can be incubated at 25° C. for 16 hours. The reaction can be stopped by heating at 65° C. for 20 minutes.

In some embodiments, the ligation reaction can be conducted using a T4 RNA ligase 2 Truncated K227Q, e.g., the T4 RNA ligase 2 Truncated K227Q from New England Biosciences, M0351S, in the following exemplary reaction mix (20 μl): T4 RNA ligase buffer (2 ul), enzyme (1 μl), PEG (10 μl), DNA (1 μl), Adenylated Adapter (0.72 μl), and water (5.28 μl). The reaction can be incubated at 25° C. for 16 hours. The reaction can be stopped by heating at 65° C. for 20 minutes.

In some embodiments, the ligation reaction can be conducted using a T4 RNA ligase 2 Truncated KQ, e.g., the T4 RNA ligase 2 Truncated KQ from New England Biosciences, M0373S, in the following exemplary reaction mix (20 μl): T4 RNA ligase buffer (2 μl), enzyme (1 μl), PEG (10 μl), DNA (1 μl), Adenylated Adapter (0.72 μl), and water (5.28 μl). The reaction can be incubated at 25° C. for 16 hours. The reaction can be stopped by heating at 65° C. for 20 minutes.

In some embodiments, the ligation reaction can be conducted using a T4 DNA ligase, e.g., the T4 DNA ligase from New England Biosciences, M0202S, in the following exemplary reaction mix (20 μl): T4 RNA ligase buffer (2 μl), enzyme (1 μl), PEG (10 μl), DNA (1 μl), Adenylated Adapter (0.72 μl), and water (5.28 μl). The reaction can be incubated at 16° C. for 16 hours. The reaction can be stopped by heating at 65° C. for 10 minutes.

The second strand synthesis step can be conducted using any suitable enzyme. For example, the second strand synthesis step can be conducted using Bst polymerase, e.g., New England Biosciences, M0275S or Klenow fragment (3′->5′ exo-), e.g., New England Biosciences, M0212S.

In some embodiments, the second strand synthesis step can be conducted using Bst polymerase, e.g., New England Biosciences, M0275S, in the following exemplary reaction mix (10 μl): water (1.5 μl), primer (0.5 μl), dNTP (1 μl), ThermoPol Reaction buffer (5 μl), and Bst (2 μl). The reaction can be incubated at 62° C. for 2 minutes and at 65° C. for 30 minutes. After the reaction, the double stranded DNA molecules are further purified.

In some embodiments, the second strand synthesis step can be conducted using Klenow fragment (3′->5′ exo-), e.g., New England Biosciences, M0212S, in the following exemplary reaction mix (10 μl): water (0.5 μl), primer (0.5 μl), dNTP (1 μl), NEB buffer (2 μl), and exo-(3 μl). The reaction can be incubated at 37° C. for 5 minutes and at 75° C. for 20 minutes. After the reaction, the double stranded DNA molecules are further purified.

After the second strand synthesis, but before the first or semi-targeted PCR, the double stranded DNA can be purified. The double stranded DNA can be purified using any suitable technique or procedure. For example, the double stranded DNA can be purified using any of the following kits: Zymo clean and concentrator, Zymo research, D4103; Qiaquick, Qiagen, 28104; Zymo ssDNA purification kit, Zymo Research, D7010; Zymo Oligo purification kit, Zymo Research, D4060; and AmpureXP beads, Beckman Coulter, A63882: 1.2×-4× bead ratio.

The first or semi-targeted PCR can be conducted using any suitable enzyme or reaction conditions. For example, the polynucleotides or DNA strands can be annealed at a temperature ranging from about 52° C. to about 72° C., e.g., at about 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., or 72° C., or any subrange thereof. The first or semi-targeted PCR can be conducted for any suitable rounds of cycles. For example, the first or semi-targeted PCR can be conducted for 10-40 cycles, e.g., for 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 cycles. The primer pool can be used at any suitable concentration. For example, the primer pool can be used at a concentration ranging from about 5 nm to about 200 nM, e.g., at about 5 nm, 6 nm, 7 nm, 8 nm, 9 nm, 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, or 200 nm, or any subrange thereof.

The first or semi-targeted PCR can be conducted using any suitable temperature cycle conditions. For example, the first or semi-targeted PCR can be conducted using any of the following cycle conditions: 95° C. 3 minutes, (95° C. 15 seconds, 62° C. 30 seconds, 72° C. 90 seconds)×3 or ×5; or (95° C. 15 seconds, 72° C. 90 seconds)×23 or ×21, 72C 1 minute, 4° C. forever.

In some embodiments, the first or semi-targeted PCR can be conducted using KAPA SYBR FAST, e.g., KAPA biosciences, KK4600, in the following exemplary reaction mix (50 μl): DNA (2 μl), KAPASYBR (25 μl), Primer Pool (26 nM each) (10 μl), Aprimer (100 μM) (0.4 ul), and water (12.6 μl). The first or semi-targeted PCR can be conducted using any of the following cycle conditions: 95° C. 30 seconds, (95° C. 10 seconds, 50-56° C. 45 seconds, 72° C. 35 seconds)×40.

In some embodiments, the first or semi-targeted PCR can be conducted using KAPA HiFi, e.g., KAPA Biosciences, KK2601, in the following exemplary reaction mix (50 μl): DNA (15 μl), KAPAHiFi (25 μl), Primer Pool (26 nM each) (10 μl), and Aprimer (100 μM) (0.4 μl). The first or semi-targeted PCR can be conducted using any of the following cycle conditions: 95° C. 3 min, (98° C. 20 seconds, 53-54° C. 15 seconds, 72° C. 35 seconds)×15, 72° C. 2 minutes, 4° C. forever.

Bisulfite conversion can be conducted using any suitable techniques, procedures or reagents. In some embodiments, bisulfite conversion can be conducted using any of the following kits and procedures provided in the kit: EpiMark Bisulfite Conversion Kit, New England Biosciences, E3318S; EZ DNA Methylation Kit, Zymo Research, D5001; MethylCode Bisulfite Conversion Kit, Thermo Fisher Scientific, MECOV50; EZ DNA Methylation Gold Kit, Zymo Research, D5005; EZ DNA Methylation Direct Kit, Zymo Research, D5020; EZ DNA Methylation Lightning Kit, Zymo Research, D5030T; EpiJET Bisulfite Conversion Kit, Thermo Fisher Scientific, K1461; or EpiTect Bisulfite Kit, Qiagen, 59104.

In some embodiments, DNA molecules can be prepared using the procedures illustrated in Example 3, including the steps for constructing single-stranded polynucleotide, conversion of single-stranded polynucleotide library to double-stranded polynucleotide library, semi-targeted amplification of double-stranded polynucleotide library, and construction of sequence library. Such DNA molecules can further be analyzed for methylation status using any suitable methods or procedures.

### G. EXAMPLES

### Example 1. Detection of Colorectal Cancer in Plasma

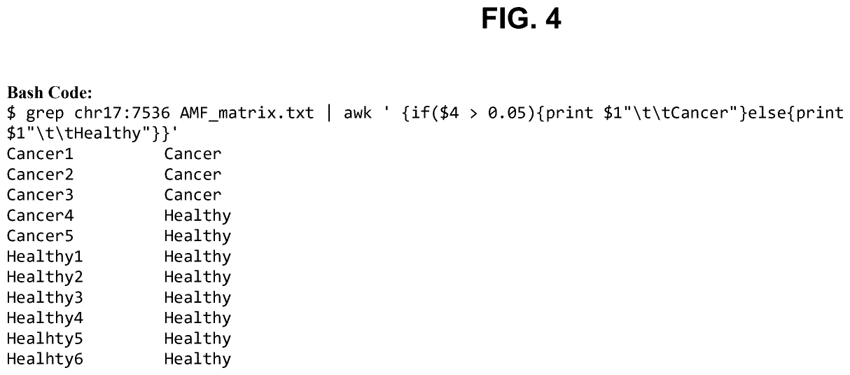

In this example, methylation status of one Target Region listed in Table 1: Target 36, was analyzed. Methylation metric is computed as Methylation Frequency (AMF). The classification method used in the analysis is Cutoff. Cutoff refers to comparing a methylation metric to a fixed pre-determined cutoff value, and calling a sample as positive if the metric exceeds this value and negative if the metric does not exceed this value. For this example of using only one target, the AMF metric was compared to a fixed value (in this case 5%). The Bash Code used in this example is illustrated in FIG. 4.

An experimental panel interrogating one target region listed above was developed. Six (6) healthy plasma and six (6) colon cancer plasma samples were processed using the experimental panel, and the AMF metric was computed for the target region to create a data matrix. A Cutoff approach was fit to the average methylation value across the target region. The cancer status of the samples was predicted by observing if >5% average methylation was observed in the target region; this led to a sensitivity of 50% for cancer plasma samples and a specificity of 100% in the healthy plasma samples.

### Example 2. Detection of Colorectal Cancer in Plasma

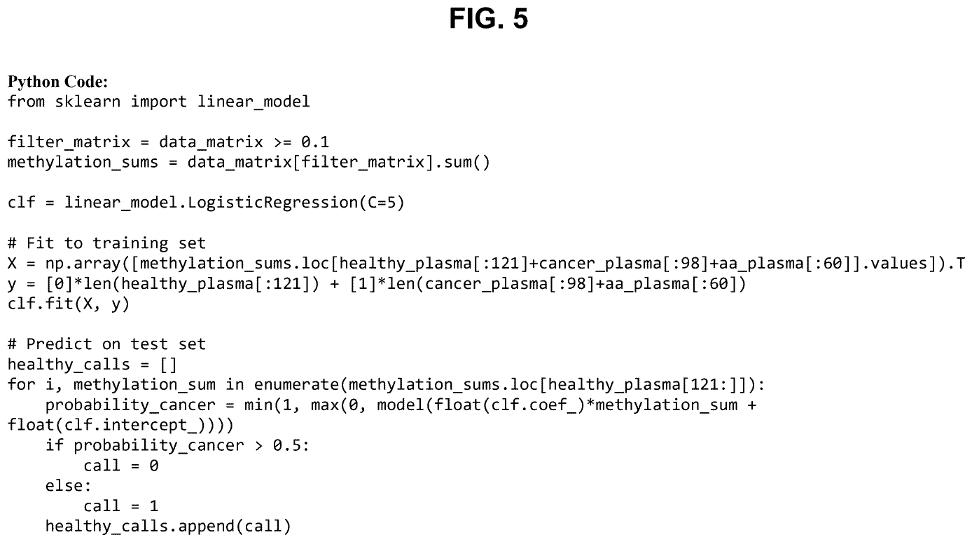

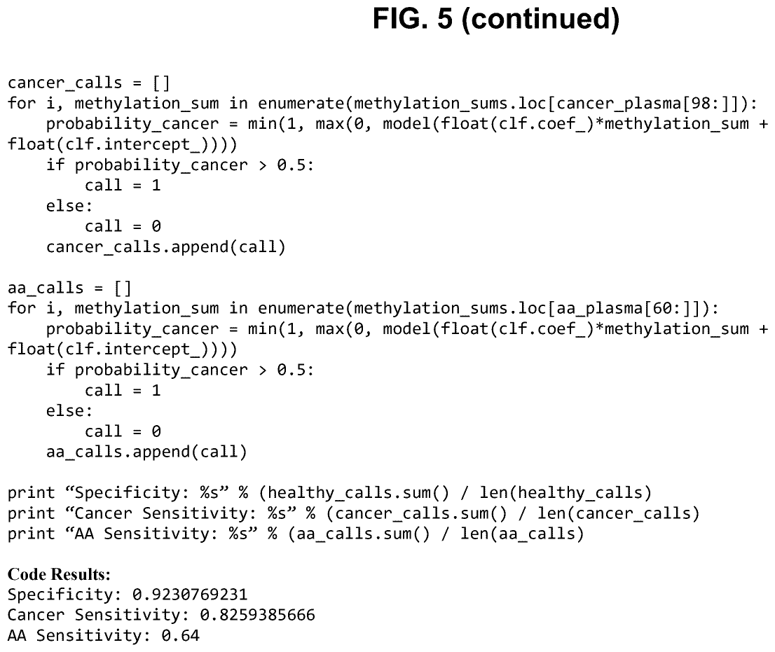

In this example, methylation status of 83 Target Regions listed in Table 1: Target1, Target2, Target3, Target4, Target5, Target6, Target7, Target8, Target9, Target10, Target11, Target12, Target13, Target14, Target15, Target16, Target17, Target18, Target19, Target20, Target21, Target22, Target23, Target24, Target25, Target26, Target27, Target28, Target29, Target30, Target31, Target32, Target33, Target34, Target35, Target36, Target37, Target38, Target39, Target40, Target41, Target42, Target43, Target44, Target45, Target46, Target47, Target48, Target49, Target50, Target51, Target52, Target53, Target54, Target55, Target56, Target57, Target58, Target59, Target60, Target61, Target62, Target63, Target64, Target65, Target66, Target67, Target68, Target69, Target70, Target71, Target72, Target73, Target74, Target75, Target76, Target77, Target78, Target79, Target80, Target81, Target82, Target83, was analyzed. Methylation metric is computed as Methylation Haplotype Load (MHL), UnMethylation Haplotype Load (UMHL), and Percent Discordant Reads (PDR). The classification method used in the analysis is logistic regression. The Python Code used in this example is illustrated in FIG. 5.

An experimental panel interrogating the 83 target regions listed above was developed. Four hundred and forty nine (449) healthy plasma, eighty five (85) advanced adenoma plasma, and three hundred and ninety one (391) colon cancer plasma samples were processed using the experimental panel, and the MHL, UMHL, and PDR metrics were computed for each target region to create a data matrix. In order to develop a Logistic Regression-based classifier, a subset of 124 healthy, 60 advanced adenoma, and 98 cancer plasma samples were selected as the “training” set. A Logistic Regression model was fit to the sum of the average methylation values across all target regions in Python (using the sklearn module). The cancer status of the remaining test set samples was then predicted; this led to a sensitivity of 64% for advanced adenoma plasma samples, a sensitivity of 83% in the cancer plasma samples, and a specificity of 92% in the healthy plasma samples.

### Example 3

In this example, the templates (e.g., polynucleotides to be sequenced) are short DNA fragments less than about 200 bp long. These DNA fragments can include extracted DNA from plasma, enzyme-treated (e.g., by a fragmentase) genomic DNA, or physically sheared DNA. The physically sheared DNA may be end repaired. In particular aspects, the template has a 3′ hydroxyl group for ligation.

Typically, 10-30 ng of the properly prepared template DNA was dephosphorylated, for example, using 1 U FastAP Thermosensitive Alkaline Phosphatase (Thermo Scientific) in 100 mM MOPS (pH 7.5), 20 mM KCl, 10 mM MgCl₂, 2 mM DTT, and 5 mM MnCl₂ at 37° C. for 10 minutes. The DNA was then denatured, for example, at 95° C. for 2 minutes and put on ice for 1 minute.

A single-stranded adapter was synthesized from IDT with a 5′ phosphate group and a 3′ carbon spacer. The 5′ end contains GA following by a 12-mer unique molecular identifier (UMI) sequence. A typical single-stranded adapter has the following sequence:/5Phos/GANNNNNNNNNNNNAGATCGGAAGAGCGTCGTGTAGGGAAAGAGTG/3SpC3/(“5Phos” represents a 5′ phosphate group, “NNNNNNNNNNNN” represents a 12-mer UMI sequence, and “3SpC3” represents a 3′ carbon spacer.

A ligation reaction was then performed using the dephosphorylated, single-stranded DNA as template. The following final concentrations were used in the ligation reaction: 50 mM MOPS (pH 7.5), 10 mM KCl, 5 mM MgCl₂, 1 mM DTT, and 2.5 mM MnCl₂, 50% PEG 4000, 0.5 μM adapter, 125 μM ATP, and 200 U Epicentre Circligase™. The reaction was incubated at 60° C. for 2 hours, 80° C. for 10 minutes, 85° C. for 2 minutes, and held at 4° C.

The DNA was then double-stranded by adding the previous reaction volume to the following: 10 mM Tris-HCl (pH 8.3), 50 mM KCl, 1.5 mM MgCl₂, 1.25 U Taq DNA Polymerase (NEB), 1 μM reverse-complement primer (a primer that is reverse-complement to the adpator), and 200 μM dNTP mix. The reaction was incubated at 95° C. for 30 seconds, 62° C. for 2 minutes, 68° C. for 10 minutes, and held at 4° C. A typical reverse-complement primer comprises the sequence set forth in SEQ ID NO:84, CACTCTTTCCCTACACGACGC (5′ to 3′). The following is an alignment between the adaptor and the reverse-complement primer:

Adaptor /5Phos/GANNNNNNNNNNNNAGATCGGAAGAGCGTCGTGTAGGGAAAGAGTG/3SpC3/
::::::::
Primer CGCAGCACATCCCTTTCTCAC

The reaction was then purified using 1.6 (bead ratio)×AmPure® XP beads. The beads were added and incubated for 10 minutes. The mixtures were then transferred to a magnet for 5 minutes. The supernatant was then removed. The beads were washed 2× with 150 μL 80% ethanol for 30 seconds each. All residual ethanol was then removed and the beads were dried for 3 minutes at room temperature off of the magnet. 15 μl of Low TE buffer (Thermo Fisher) was added to the beads and incubated for 2 minutes. The beads were then returned to the magnet for 1 minute. The supernatant was removed and stored for the next reaction. In one aspect, the bead ratio causes size selectivity in the purification process, and a bead ratio (such as 1.6×) can be selected that removes molecules shorter than about 100 bp.

A set of PCR primers were designed to minimize primer-primer interactions and off-target annealing. The primers were further optimized to land within close proximity to specific variants. Once designed, the primers were synthesized by IDT. The primers were mixed in equal volume ratios into a primer pool. A semi-targeted PCR reaction was performed with the following reagents: all purified DNA from previous reaction, 1×KAPA 2G multiplex master mix, 66 nM of each primer from pool, and 800 nM reverse-complement primer. The reaction underwent the following thermo-cycling program: 95° C. 3 minutes, (95° C. 15 seconds, 72° C. 90 seconds)×20, 72° C. 1 minute, and held at 4° C.

The reaction was then purified using 1.6 (bead ratio)×AmPure® XP beads. The beads were added and incubated for 10 minutes. The mixtures were then transferred to a magnet for 5 minutes. The supernatant was then removed. The beads were washed 2× with 150 μL 80% ethanol for 30 seconds each. All residual ethanol was then removed and the beads were dried for 3 minutes at room temperature off of the magnet. 20 μl of Low TE buffer (Thermo Fisher) was added to the beads and incubated for 2 minutes. The beads were then returned to the magnet for 1 minute. The supernatant is removed and stored for the next reaction. A bead ratio (such as 1.6×) can be selected that removes molecules shorter than about 100 bp, including free adaptor molecules, free primer molecules, and/or adaptor/primer dimers.

Another PCR reaction was then completed to add full length sequencing adapters and sample specific barcodes. The PCR reaction contained the following: 2 μL purified DNA from previous reaction, 1× NEB ultra Q5 II master mix, 400 nM universal primer, and 400 nM barcode specific primer. The reaction underwent the following thermo-cycling program: 95° C. 3 minutes, (98° C. 10 seconds, 65° C. 75 seconds)×10, 65° C. 2 minute, and held at 4° C.

The reaction was then purified using 0.8 (bead ratio)×AmPure® XP beads. The beads were added and incubated for 10 minutes. The mixtures were then transferred to a magnet for 5 minutes. The supernatant was then removed. The beads were washed 2× with 150 μL 80% ethanol for 30 seconds each. All residual ethanol was then removed and the beads were dried for 3 minutes at room temperature off of the magnet. 25 μl of Low TE buffer (Thermo Fisher) is added to the beads and incubated for 2 minutes. The beads were then returned to the magnet for 1 minute. The supernatant is removed and is ready for sequencing. A bead ratio (such as 0.8×) can be selected that removes a majority of molecules shorter than about 200 bp.

### Example 4. Detection of Colorectal Cancer in Tissue

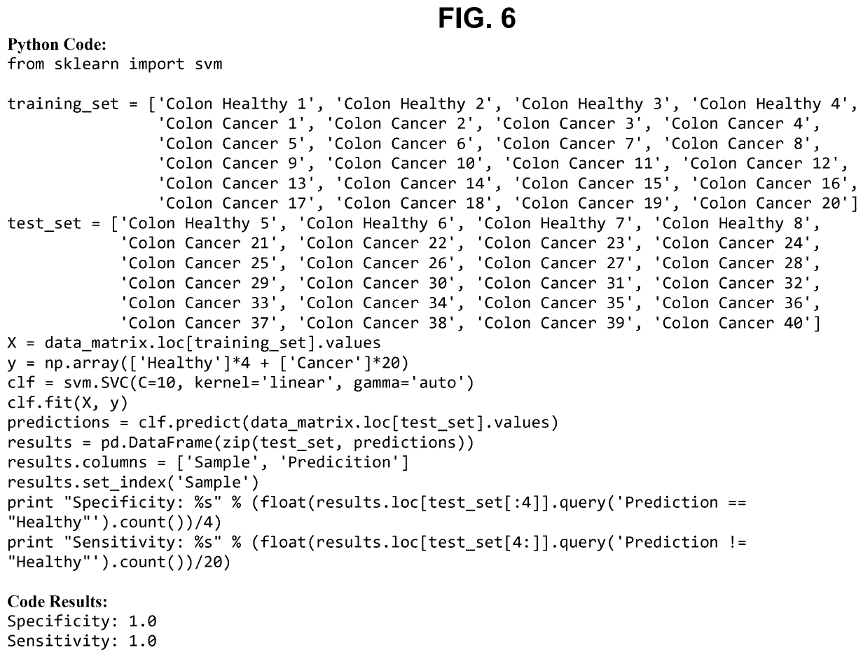

In this example, methylation status of 10 Target Regions listed in Table 1: Target6, Target8, Target18, Target25, Target35, Target41, Target43, Target54, Target74, Target82, was analyzed. Methylation metric is computed as UnMethylation Haplotype Load (UMHL). The classification method used in the analysis is Support Vector Machines. The Python Code used in this example is illustrated in FIG. 6.

An experimental panel interrogating the 10 target regions listed above was developed. 8 healthy colon and 40 colon cancer tissue samples were processed using the experimental panel, and the uMHL metric was computed for each target region to create a data matrix. In order to develop a SVM-based classifier, a subset of 4 healthy colon and 20 colon cancer tissue samples were selected as the “training” set. The training set samples were used to fit an SVM-based classifier in Python (using the scikit-learn library). The SVM-based classifier was then used to predict the categories for the remaining 24 “test” set samples. The classifier was able to achieve 100% accuracy in the test set and correctly identify if a colon tissue sample was cancerous.

### Example 5. Detection of Colorectal Cancer in Plasma

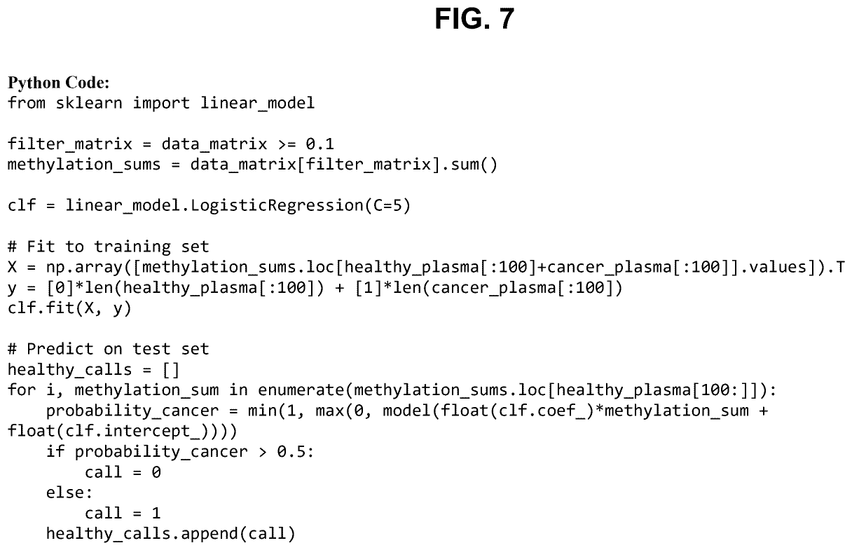

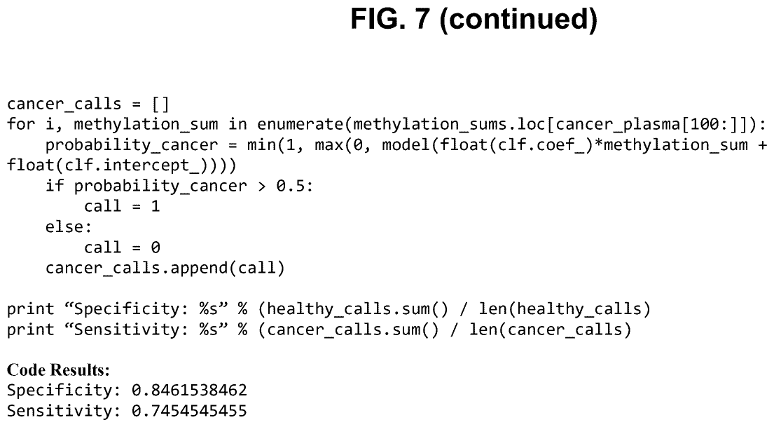

In this example, methylation status of 36 Target Regions listed in Table 1: Target1, Target2, Target6, Target8, Target10, Target12, Target15, Target16, Target19, Target20, Target27, Target28, Target31, Target32, Target34, Target39, Target40, Target41, Target42, Target43, Target45, Target46, Target49, Target55, Target57, Target58, Target61, Target64, Target66, Target68, Target70, Target71, Target72, Target74, Target76, Target79, was analyzed. Methylation metric is computed as Methylation Haplotype Load (MHL), UnMethylation Haplotype Load (UMHL), and/or Percent Discordant Reads (PDR). The classification method used in the analysis is Logistic Regression. The Python Code used in this example is illustrated in FIG. 7.

An experimental panel interrogating the 36 target regions listed above was developed. 139 healthy plasma and 155 colon cancer plasma samples were processed using the experimental panel, and the MHL, UMHL, and PDR metrics were computed for each target region to create a data matrix. In order to develop a Logistic Regression-based classifier, a subset of 100 healthy, and 100 cancer plasma samples were selected as the “training” set. A Logistic Regression model was fit to the sum of the average methylation values across all target regions in Python (using the sklearn module). The cancer status of the remaining test set samples was then predicted; this led to a sensitivity of 74% in the cancer plasma samples and a specificity of 85% in the healthy plasma samples.

1. A panel of isolated polynucleotides comprising, consisting of, or consisting essentially of one or at least two isolated polynucleotides, each of said isolated polynucleotides having a polynucleotide sequence of any of Target 1 to Target 83 listed in Table 1, or a complementary or substantially complementary sequence thereof.

2-10. (canceled)

11. A kit or system, which comprises a panel of claim 1.

12. The kit or system of claim 11, which is configured for assessing methylation status of one or at least two of Target 1 to Target 83 listed in Table 1, e.g., configured for in vitro assessing of methylation status of one or at least two of Target 1 to Target 83 listed in Table 1 in one or more blood or plasma sample(s) obtained from one or more subject(s).

13-15. (canceled)

16. A kit or system, which comprises reagents for assessing methylation status of one or more of Target 1 to Target 83 listed in Table 1.

17-45. (canceled)

46. The kit or system of claim 6, which further comprises a computer readable medium that is configured for obtaining a methylation metric in the form of average methylation frequency, methylation haplotype load, unmethylation haplotype load, percent discordant reads, or a combination thereof.

47. The kit or system of claim 16, which is configured for assessing colorectal cancer and/or advanced adenoma in a subject.

48. An in vitro method for classifying a blood sample, which method comprises:
a) in vitro assessing methylation status of one or at least two target polynucleotides in a blood sample from a subject, each of said target polynucleotides having a polynucleotide sequence of any of Target 1 to Target 83 listed in Table 1, or a complementary or substantially complementary sequence thereof; and
b) classifying said blood sample into a category based on said methylation status of said one or at least two target polynucleotides in said blood sample obtained in a).

49. (canceled)

50. The method of claim 48, which is used for assessing colorectal cancer and/or advanced adenoma in a subject.

51-55. (canceled)

56. The method of claim 48, which comprises assessing methylation status of target polynucleotides having polynucleotide sequences of at least 2, 3, 4, 5, 6, 7, 8, 9, 10, 20, 30, 40, 50, 60, 70, 80, or 83 of the isolated polynucleotides, or the isolated polynucleotides corresponding to all of Target 1 to Target 83 listed in Table 1.

57-62. (canceled)

63. The method of claim 48 which further comprises isolating the target polynucleotides from a blood sample.

64. The method of claim 48 which further comprises preparing a library of the target polynucleotides.

65-66. (canceled)

67. The method of claim 48 which further comprises amplifying the target polynucleotide(s).

68. (canceled)

69. The method of claim 48 which further comprises purifying the target polynucleotide(s), a library of the target polynucleotides, amplified target polynucleotide(s) or a library of amplified target polynucleotides.

70. The method of claim 48, wherein the methylation status of target polynucleotide(s) is assessed using mass spectrometry, methylation-specific PCR (MSP), methylation-sensitive sequencing, e.g., bisulphite sequencing, the HpaII tiny fragment Enrichment by ligation-mediated PCR assay (HELP Assay), Glal hydrolysis and ligation adapter dependent PCR assay (GLAD-PCR assay), restriction landmark genomic scanning (RLGS), methylated DNA immunoprecipitation (MeDIP or mDIP), pyrosequencing, molecular break light assay for DNA adenine methyltransferase activity, methyl sensitive Southern blotting or high resolution Melt (HRM) analysis.

71. (canceled)

72. The method of claim 70, wherein assessing methylation status of target polynucleotide(s) comprises using a biological agent, e.g., a polypeptide or an enzyme.

73-79. (canceled)

80. The method of claim 48, wherein the methylation status of target polynucleotide(s) is assessed using methylation-sensitive sequencing, e.g., bisulphite sequencing.

81. (canceled)

82. The method of claim 80, which further comprises, prior to the methylation-sensitive sequencing, e.g., bisulphite sequencing, obtaining a library of linear, single-stranded ligation products, each of the linear, single-stranded ligation products comprises of a linear, single-stranded target polynucleotide linked to an adaptor comprising a unique molecular identifier (UMI) sequence that earmarks the single-stranded target polynucleotide to which the adaptor is ligated.

83-85. (canceled)

86. The method of claim 48, wherein methylation status of each of the target polynucleotides is assessed.

87. The method of claim 86, wherein the methylation status of each of the target polynucleotides is assessed to obtain a methylation metric, e.g., in the form of average methylation frequency, methylation haplotype load, unmethylation haplotype load, percent discordant reads, or a combination thereof.

88-92. (canceled)

93. The method of claim 87, wherein the numerical methylation matrix is computed using a classification algorithm from the methylation metrics from each of the target polynucleotides to assess methylation status of a sample.

94-98. (canceled)

99. The method of claim 50, which is used for diagnosis, prognosis, stratification, risk assessment, or treatment monitoring of colorectal cancer and/or advanced adenoma in a subject.

100-101. (canceled)

102. The method of claim 50, wherein colorectal cancer or advanced adenoma in multiple subjects are assessed sequentially or simultaneously.

103-104. (canceled)

105. The method of claim 50, which further comprises treating a subject or altering treatment of a subject based on the assessment of colorectal cancer and/or advanced adenoma in the subject.

106-107. (canceled)


SEQUENCE LISTING (86 sequences)

SEQ ID NO: 1 - DNA, 27 bp, Homo sapiens
  source 1..27: mol_type = genomic DNA
ttttggttgtgtggattgggtttgtgg

SEQ ID NO: 2 - DNA, 27 bp, Homo sapiens
  source 1..27: mol_type = genomic DNA
caaaaacaaaacaacacaccctctcca

SEQ ID NO: 3 - DNA, 27 bp, Homo sapiens
  source 1..27: mol_type = genomic DNA
agtgtggttttgatagcggggaggggg

SEQ ID NO: 4 - DNA, 27 bp, Homo sapiens
  source 1..27: mol_type = genomic DNA
gcgggaggaggttcgtgggatgttgta

SEQ ID NO: 5 - DNA, 27 bp, Homo sapiens
  source 1..27: mol_type = genomic DNA
aaagggggttttgtgttgcgtcgggag

SEQ ID NO: 6 - DNA, 27 bp, Homo sapiens
  source 1..27: mol_type = genomic DNA
ttttgttgtcgggagtcgggggttgga

SEQ ID NO: 7 - DNA, 27 bp, Homo sapiens
  source 1..27: mol_type = genomic DNA
gttgttaggggaagggggcgttagggt

SEQ ID NO: 8 - DNA, 27 bp, Homo sapiens
  source 1..27: mol_type = genomic DNA
ggggttgtatttgtggttgcggggtcg

SEQ ID NO: 9 - DNA, 27 bp, Homo sapiens
  source 1..27: mol_type = genomic DNA
agggagatttggggggttggatgtgtt

SEQ ID NO: 10 - DNA, 27 bp, Homo sapiens
  source 1..27: mol_type = genomic DNA
ggaagcgggaggtattgggggtgttga

SEQ ID NO: 11 - DNA, 27 bp, Homo sapiens
  source 1..27: mol_type = genomic DNA
atggggtgggcgttagtgtaggttggg

SEQ ID NO: 12 - DNA, 27 bp, Homo sapiens
  source 1..27: mol_type = genomic DNA
gtggattcgggatggggaggaggttgg

SEQ ID NO: 13 - DNA, 27 bp, Homo sapiens
  source 1..27: mol_type = genomic DNA
atatgagtgcggggcgtcggtgagttt

SEQ ID NO: 14 - DNA, 27 bp, Homo sapiens
  source 1..27: mol_type = genomic DNA
aggtttgtgttaggcgtgggggtaggg

SEQ ID NO: 15 - DNA, 27 bp, Homo sapiens
  source 1..27: mol_type = genomic DNA
ggtagtttcgcggggttgggagtggat

SEQ ID NO: 16 - DNA, 27 bp, Homo sapiens
  source 1..27: mol_type = genomic DNA
ggttgggtttggggaggtgatttgggc

SEQ ID NO: 17 - DNA, 27 bp, Homo sapiens
  source 1..27: mol_type = genomic DNA
gtggggacgttgagcggtcgttaggat

SEQ ID NO: 18 - DNA, 27 bp, Homo sapiens
  source 1..27: mol_type = genomic DNA
tatggagtgttgaggggaggggggagt

SEQ ID NO: 19 - DNA, 27 bp, Homo sapiens
  source 1..27: mol_type = genomic DNA
gggggttttggttagttcgggcgttcg

SEQ ID NO: 20 - DNA, 27 bp, Homo sapiens
  source 1..27: mol_type = genomic DNA
cggtttgtagggtgagttacgcgggga

SEQ ID NO: 21 - DNA, 27 bp, Homo sapiens
  source 1..27: mol_type = genomic DNA
ggcgtaggaaggggaggtttcgggaag

SEQ ID NO: 22 - DNA, 27 bp, Homo sapiens
  source 1..27: mol_type = genomic DNA
ggggatgtggggaagtggttagtgggt

SEQ ID NO: 23 - DNA, 27 bp, Homo sapiens
  source 1..27: mol_type = genomic DNA
taaagtcggggttatcgtggggtgggc

SEQ ID NO: 24 - DNA, 27 bp, Homo sapiens
  source 1..27: mol_type = genomic DNA
gggtcgttagcggaggcgatggttagt

SEQ ID NO: 25 - DNA, 27 bp, Homo sapiens
  source 1..27: mol_type = genomic DNA
gcgggtgagaggggtcggtagttgttt

SEQ ID NO: 26 - DNA, 27 bp, Homo sapiens
  source 1..27: mol_type = genomic DNA
taggtcggggtttagggcgttgggttg

SEQ ID NO: 27 - DNA, 27 bp, Homo sapiens
  source 1..27: mol_type = genomic DNA
agtagggttgggggtttcggattgcgg

SEQ ID NO: 28 - DNA, 28 bp, Homo sapiens
  source 1..28: mol_type = genomic DNA
tgggggttttcggggtagagatgtgagt

SEQ ID NO: 29 - DNA, 29 bp, Homo sapiens
  source 1..29: mol_type = genomic DNA
tggaatttgggtttatgggggattcgggt

SEQ ID NO: 30 - DNA, 27 bp, Homo sapiens
  source 1..27: mol_type = genomic DNA
taatggtagggatggtttggggcgcga

SEQ ID NO: 31 - DNA, 27 bp, Homo sapiens
  source 1..27: mol_type = genomic DNA
ggggggcgggttaaagtgagaatggga

SEQ ID NO: 32 - DNA, 27 bp, Homo sapiens
  source 1..27: mol_type = genomic DNA
gtcgggggttatggttttcgagggggt

SEQ ID NO: 33 - DNA, 27 bp, Homo sapiens
  source 1..27: mol_type = genomic DNA
ggtggtaagcggagggtgggtttggta

SEQ ID NO: 34 - DNA, 27 bp, Homo sapiens
  source 1..27: mol_type = genomic DNA
ggagggaggtcggagtggagtagggat

SEQ ID NO: 35 - DNA, 27 bp, Homo sapiens
  source 1..27: mol_type = genomic DNA
gggagggcgtttggattaggggtttcg

SEQ ID NO: 36 - DNA, 27 bp, Homo sapiens
  source 1..27: mol_type = genomic DNA
tcgaaggtgggtgttgggttggttgtt

SEQ ID NO: 37 - DNA, 27 bp, Homo sapiens
  source 1..27: mol_type = genomic DNA
tttggcgtttgttttggggagggacgg

SEQ ID NO: 38 - DNA, 27 bp, Homo sapiens
  source 1..27: mol_type = genomic DNA
gagtcgtggcggttggtggcgttgtag

SEQ ID NO: 39 - DNA, 27 bp, Homo sapiens
  source 1..27: mol_type = genomic DNA
aaagttgatgatgacgggttcgggggc

SEQ ID NO: 40 - DNA, 27 bp, Homo sapiens
  source 1..27: mol_type = genomic DNA
aatggagcgtcggtatggggttgggtt

SEQ ID NO: 41 - DNA, 27 bp, Homo sapiens
  source 1..27: mol_type = genomic DNA
gttcgaagagggagaggggttgtcggg

SEQ ID NO: 42 - DNA, 27 bp, Homo sapiens
  source 1..27: mol_type = genomic DNA
ggaaagggcggtggttgtgggtaaagc

SEQ ID NO: 43 - DNA, 27 bp, Homo sapiens
  source 1..27: mol_type = genomic DNA
ggtgtgtttgaggaggtgggtgggagt

SEQ ID NO: 44 - DNA, 27 bp, Homo sapiens
  source 1..27: mol_type = genomic DNA
tgttggttgggggcgattgaggaggag

SEQ ID NO: 45 - DNA, 27 bp, Homo sapiens
  source 1..27: mol_type = genomic DNA
ttaggggagttgatggggttggtggcg

SEQ ID NO: 46 - DNA, 27 bp, Homo sapiens
  source 1..27: mol_type = genomic DNA
tgcgggtggggacgtattgagaggttc

SEQ ID NO: 47 - DNA, 27 bp, Homo sapiens
  source 1..27: mol_type = genomic DNA
gttggagggggagtttcgggttttcgg

SEQ ID NO: 48 - DNA, 27 bp, Homo sapiens
  source 1..27: mol_type = genomic DNA
gttttgggatttcgtcggggcgttggt

SEQ ID NO: 49 - DNA, 27 bp, Homo sapiens
  source 1..27: mol_type = genomic DNA
agcggaagtagcgtggatttgtagggg

SEQ ID NO: 50 - DNA, 27 bp, Homo sapiens
  source 1..27: mol_type = genomic DNA
tttgggagtgggagtggggagagttgc

SEQ ID NO: 51 - DNA, 28 bp, Homo sapiens
  source 1..28: mol_type = genomic DNA
gggcgttcgaaggtttatacggtgaggt

SEQ ID NO: 52 - DNA, 27 bp, Homo sapiens
  source 1..27: mol_type = genomic DNA
atttttgggtggagagggttgcgggga

SEQ ID NO: 53 - DNA, 27 bp, Homo sapiens
  source 1..27: mol_type = genomic DNA
gtttagtagggggcgaggggtgttggg

SEQ ID NO: 54 - DNA, 27 bp, Homo sapiens
  source 1..27: mol_type = genomic DNA
cgggttagtcggggttggagttgggaa

SEQ ID NO: 55 - DNA, 27 bp, Homo sapiens
  source 1..27: mol_type = genomic DNA
atgtcgggttaggggaagtcgaaggcg

SEQ ID NO: 56 - DNA, 27 bp, Homo sapiens
  source 1..27: mol_type = genomic DNA
tggggtgttggttgaggttaaggcggt

SEQ ID NO: 57 - DNA, 27 bp, Homo sapiens
  source 1..27: mol_type = genomic DNA
cgggagggagtaagggatggagttgcg

SEQ ID NO: 58 - DNA, 27 bp, Homo sapiens
  source 1..27: mol_type = genomic DNA
aggtgggattttgtgcggggggtagag

SEQ ID NO: 59 - DNA, 27 bp, Homo sapiens
  source 1..27: mol_type = genomic DNA
aagagatcgacggggtgagtggtgtgg

SEQ ID NO: 60 - DNA, 31 bp, Homo sapiens
  source 1..31: mol_type = genomic DNA
cggaggttgttagtggaaagtttgagggggg

SEQ ID NO: 61 - DNA, 27 bp, Homo sapiens
  source 1..27: mol_type = genomic DNA
gggttgcgttgggggttttatagggcg

SEQ ID NO: 62 - DNA, 27 bp, Homo sapiens
  source 1..27: mol_type = genomic DNA
gggggacgtggtggttaggatcgtgtt

SEQ ID NO: 63 - DNA, 29 bp, Homo sapiens
  source 1..29: mol_type = genomic DNA
gcgggttttgagtagtcgttttgtatggt

SEQ ID NO: 64 - DNA, 27 bp, Homo sapiens
  source 1..27: mol_type = genomic DNA
gggatcgcgcgggatggaggtagttaa

SEQ ID NO: 65 - DNA, 27 bp, Homo sapiens
  source 1..27: mol_type = genomic DNA
tgaaggtgtgggttggtgggttagggg

SEQ ID NO: 66 - DNA, 27 bp, Homo sapiens
  source 1..27: mol_type = genomic DNA
gtttggttcggttttggcgaagggggt

SEQ ID NO: 67 - DNA, 27 bp, Homo sapiens
  source 1..27: mol_type = genomic DNA
aaaggcggattgagggtggaggaggtc

SEQ ID NO: 68 - DNA, 27 bp, Homo sapiens
  source 1..27: mol_type = genomic DNA
cggggaagcgggggagaaagtttaggt

SEQ ID NO: 69 - DNA, 27 bp, Homo sapiens
  source 1..27: mol_type = genomic DNA
gaagggggtggaggtgacggggtttag

SEQ ID NO: 70 - DNA, 27 bp, Homo sapiens
  source 1..27: mol_type = genomic DNA
tagggcgtgggtagtggtggtagtgga

SEQ ID NO: 71 - DNA, 27 bp, Homo sapiens
  source 1..27: mol_type = genomic DNA
agagaggtatcgggcgagaaggggtgt

SEQ ID NO: 72 - DNA, 27 bp, Homo sapiens
  source 1..27: mol_type = genomic DNA
aggcggatgaattcggttggagagggg

SEQ ID NO: 73 - DNA, 27 bp, Homo sapiens
  source 1..27: mol_type = genomic DNA
gggagtaggcgtaggaggaggaagcga

SEQ ID NO: 74 - DNA, 27 bp, Homo sapiens
  source 1..27: mol_type = genomic DNA
ggtggttttgtcgtggatgtgggcgtt

SEQ ID NO: 75 - DNA, 27 bp, Homo sapiens
  source 1..27: mol_type = genomic DNA
cgttagtcgggttgggttcgggaggag

SEQ ID NO: 76 - DNA, 27 bp, Homo sapiens
  source 1..27: mol_type = genomic DNA
gatggtgagcgaagaggcggaggagtt

SEQ ID NO: 77 - DNA, 27 bp, Homo sapiens
  source 1..27: mol_type = genomic DNA
gaaggtagtgtggcgcgtgcgtattgg

SEQ ID NO: 78 - DNA, 27 bp, Homo sapiens
  source 1..27: mol_type = genomic DNA
gaggttagcgtcggtcgtttttcgcgg

SEQ ID NO: 79 - DNA, 27 bp, Homo sapiens
  source 1..27: mol_type = genomic DNA
gcggtgtcgggtttggggttagtttcg

SEQ ID NO: 80 - DNA, 27 bp, Homo sapiens
  source 1..27: mol_type = genomic DNA
tcggagggcgtagtagaggttggggtt

SEQ ID NO: 81 - DNA, 27 bp, Homo sapiens
  source 1..27: mol_type = genomic DNA
gggggagggggtagtaggtcgatcgtt

SEQ ID NO: 82 - DNA, 27 bp, Homo sapiens
  source 1..27: mol_type = genomic DNA
gggaagtagggggtaggggagaggtcg

SEQ ID NO: 83 - DNA, 27 bp, Homo sapiens
  source 1..27: mol_type = genomic DNA
ggggtatggcgggggtagtagacgttg

SEQ ID NO: 84 - DNA, 21 bp, Homo sapiens
  source 1..21: mol_type = genomic DNA
cactctttccctacacgacgc

SEQ ID NO: 85 - DNA, 32 bp, synthetic construct
  source 1..32: mol_type = other DNA
agatcggaagagcgtcgtgtagggaaagagtg

SEQ ID NO: 86 - DNA, 46 bp, synthetic construct
  source 1..46: mol_type = other DNA
gannnnnnnnnnnnagatcggaagagcgtcgtgtagggaaagagtg